(19) United States
(12) Patent Application Publication
(10) Pub. No.: US 2005/0240034 A1
(43) Pub. Date: Oct. 27, 2005

(54) Artemisinin-based peroxide compounds as broad spectrum anti-infective agents

(76) Inventors: Mitchell Avery, Oxford, MS (US); Kannoth M. Muraleedharan, Oxford, MS (US)

Correspondence Address: Eugene C Rzucidlo;Greenberg Traurig, 200 Park Avenue, 34th Floor, New York, NY, 10166, US

(21) Appl. No.: 10/514,028

(22) PCT Filed: May 7, 2003
(86) PCT No.: PCT/US03/14571

§ 371 Date: Apr. 14, 2005

Related U.S. Application Data
(60) Provisional application No. 60/378,534, filed on May 7, 2002.

(51) Int. Cl. C07D 321/12

(52) U.S. Cl. 549/349

(57) ABSTRACT

Described herein is the synthesis, bioassay results and utility of new C-9 and C-10 substituted artemisinin derivatives with easily functionalizable groups attached to the artemisinin skeleton through carbon chain or heteroatoms. Described also is the demonstration of this class of compounds for their broad-spectrum anti-parasitic activity. Certain of these analogs possess noticeable cytotoxicity deliberately focused on treatment of cancerous diseases.

This application is a continuation-in-part of U.S. provisional application Ser. No. 60/378,534 filed May 7, 2002, and the complete contents of that application are incorporated herein by reference.

Funding for the research which led to this invention was provided in part by the United States Government in CDC Grant Numbers U50/CCU418652 and UR3/CCU418652 and the government may have certain rights in this invention.

### FIELD OF THE INVENTION

This invention describes the synthesis and biological activities of analogs of artemisinin, a naturally occurring sesquiterpene endoperoxide having various anti-infective activities. The most well known effect of artemisinin in man is its antimalarial effect against Plasmodium falciparum. The analogs are unique because of their high oral potency relative to artemisinin, and low toxicity relative to marketed analogs of dihydroartemisinin.

### BACKGROUND OF THE INVENTION

The majority of the population in Third World Countries are now at the high risk of various parasitic infections such as malaria, leishmaniasis, trypanosomiasis etc., which causes more than 3 million deaths every year. Since the market for drugs against such diseases is poor in these countries, innovative and cost effective drug development programs to counter these parasitic infections are needed very urgently. The development of a broad-spectrum anti-parasitic agent that can target multiple protozoan parasites could be a significant step to reduce morbidity and mortality caused by such infections.

Malaria is a blood disease resulting from infection with a protozoan parasite known as Plasmodium.¹ Over one hundred species of the malaria parasite exist, capable of infecting various hosts such as reptiles, birds, rodents, and primates. However, only four species of the genus Plasmodium can cause human malaria. P. vivax is the most common and fatal. P. ovale and P. malariae are less common and have intermediate severity. P. falciparum is the most virulent and is responsible for the high infant mortality. The disease is transmitted to human beings through the bite of infected female Anopheles mosquitoes. It should also be noted that malaria could be transmitted by transfusion of infected blood. Unlike some infectious diseases, protective immunity is not conferred by a single episode of malaria.

Today, malaria is one of the deadliest diseases on the planet and the leading cause of sickness and death in the developing world. Four decades ago, malaria was not a real concern for most of the world: The number of people living in an at-risk area was only 10% of the world's population. Today this percentage approaches 44%, due mostly to both the emergence and spread of drug-resistant malaria parasites and pesticide-resistant malaria-transmitting mosquitoes. According to the World Health Organization (WHO), malaria causes approximately 500 million clinical cases per year and kills 2.7 million people.² It is prevalent in children, causing one million deaths in children under the age of five each year. The disease also causes anemia in children and pregnant women and increases vulnerability to other diseases. It afflicts the underprivileged most severely, decreasing productivity and causing chronic poor health. It takes approximately 15 years of continuous exposure to parasites to develop protective immunity. Consequently, children growing up in endemic areas can contract the disease before their immunity levels are sufficient.

Medicinal use of the Chinese herb qinqhao appears in several standard Chinese Materia Medica texts as a treatment for febrile illnesses.³ The herb was specifically recommended for fevers in the Zhou Hou Bei fi Fang (The Handbook of Prescriptions for Emergencies) written by Ge Heng and published in 341 AD. The most detailed description appears in the “Compendium of Materia Medica—Ben Cao Gang Mu, compiled in 1596, and is still printed in China today.^{4,5} The antimalarial activity of qinghao was rediscovered in China in 1972, and the antimalarial active principal of qinghao was named “qinghaosu”. The western name for the compound is artemisinin (1).

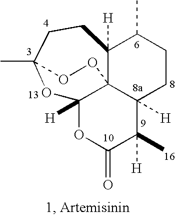

In 1834, quinine (2), an alkaloid isolated from the bark of the Cinchona tree in Peru, was introduced for the treatment of malaria. This was followed by the development of chloroquine (3), and mefloquine (4), which are very effective antimalarial drugs. But the emergence of the drug resistant strains of the parasite, coupled with potential toxicity issues limit their use.

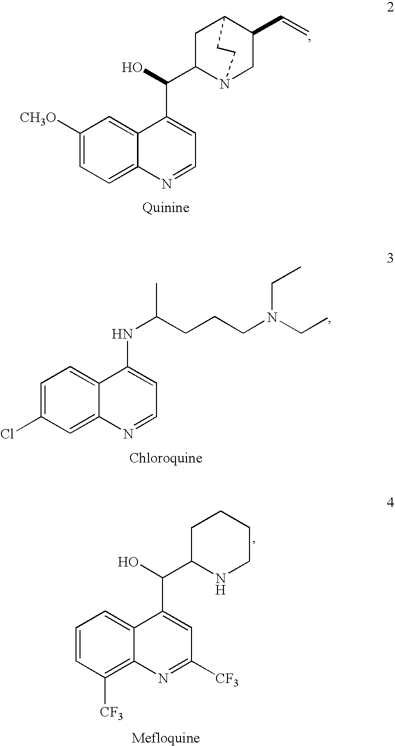

Artemisinin (1) and its related compounds are the most effective new drugs. They have been used in some areas, since they are both potent and rapidly acting antimalarials effective against chloroquine-resistant P. falciparum. The increasing resistance of malarial parasites to existing drugs highlights the continued need for new antimalarial agents.

Human leishmaniasis comprises a heterogeneous spectrum of diseases. Three major forms are generally distinguished: cutaneous leishmaniasis, mucocutaneous leishmaniasis and visceral leishmaniasis, of which the latter is potentially lethal. They are caused by various species of the protozoan parasite Leishmania and transmitted by female sandflies.⁶ The disease is currently proposed to affect some 12 million people in 88 countries.⁷⁻¹¹ It is estimated that ˜350 million people are exposed to infection by different species of Leishmania parasite. Leishmania/HIV co-infection is now considered as an ‘emerging disease’ especially in southern Europe, where 25-70% of adult visceral leishmaniasis cases are related to HIV infection.

The current treatment for leishmaniasis involves administration of pentavalent antimony complexed to a carbohydrate in the form of sodium stibogluconate (Pentosam or Sb(V)) or meglumine antimony (Glucantine), which are the only antileishmanial chemotherapeutic agents with a clearly favorable therapeutic index.^{12,13} The exact chemical structure and mode of action of pentavalent antimonials is still uncertain but, as with most metals, is thought to be multi-factorial.¹³ Amphotericin B and Pentamidine are the second line of antileishmanial agents, but are reserved for non-responding infections due to potential toxicity.¹⁴ The development of lipid-associated amphotericin B was an important progress in this area.^{15, 16} Even though many promising leads exist, none of the above mentioned drugs show promise as ideal treatment alternatives for the developing world due to their route of administration (parenteral), high clinical failure rate (40% for pentavalent antimonial agents), side effects and cost. Thus, the identification of an effective anti-leishmanial agent for oral administration would be a significant step towards reducing mortality due to leishmaniasis.

Human African trypanosomiasis (sleeping sickness) is caused by a subspecies of the parasitic haemoflagellate, Trypanosoma brucei.¹⁷⁻²⁰ The infection begins with the bite of an infected tsetse fly (Glossina spp.). Two forms of the disease are known, one caused by Trypanosoma brucei rhodesiense, endemic in Eastern and Southern Africa, and the other T. b. gambiense, originally detected in West Africa, but also widespread in Central Africa. In the former case, parasites rapidly invade the CNS causing death within weeks if untreated. The latter one proliferates relatively slowly and can take several years before infecting CNS system. There are mainly four important drugs licensed to treat these infections. Of these, pentamidine and suramin, are used before the CNS involvement. The arsenic-based drug, melarsoprol is used in the case of infections established in the CNS. The fourth drug, eflornithine, is used against late stage infection caused by T. b. gambiense. This drug is ineffective against T. b. rhodesiense. Nifurtimox is another drug licensed for both South American trypanosomiasis and melarsoprol-refractory late sage disease.

Artemisinin

Artemisinin (1) is a naturally occurring peroxidic cadinane sesquiterpene. Additional names found in China for 1 include qinghaosu, huanghuahaosu, arteannuin, and artemisinine. The Chemical Abstracts adopts artemisinin as the official name, however earlier entries as qinghaosu can be found. Systematically it is named 3,6,9-trimethyloctahydro (3a, 5aβ, 6β, 9α, 12β, 1aR)-(R)-(+)-3,12-epoxy-12H-pyrano[4,3-j]-1,2-benzodioxepin-10-(3H)one. Chinese researchers isolated artemisinin (1) in 1972 from Artemisia annua L. (annual wormwood) and its structure was elucidated in 1979.²¹ The plant now grows in many countries, although originally from northern China.²² Artemisinin (1) yields in the plant can vary considerably, depending on plant material and growth conditions. It is present in the leaves and the flowers of the plant in 0.01-0.8% dry weight.² It has shown the ability to quickly lower parasite levels, even in severe cases of cerebral malaria.

Additionally, artemisinin has demonstrated activity against drug resistant strains of P. falciparum such as W-2 Indochina (chloroquine-resistant) and D-6 Sierra Leone (mefloquine-resistant) clones. Due to this outstanding pharmacological profile in combination with its novel chemical structure, artemisinin (1) became a target studied worldwide. However, problems associated with artemisinin, including short plasma half-life, limited bioavailability, poor solubility in oil as well as water, and a low yield of artemisinin from natural sources, prompted scientists to develop new syntheses of artemisinin derivatives for more than a decade.²³ Several analogs are obtained semi-synthetically from artemisinin. For example, artemisinin can be reduced to dihydroartemisinin (5), which is approximately 10 times more potent than the parent compound in vitro. Derivatives such as artemether (6) and sodium artesunate (7), obtained by simple modification to the lactol (5), developed by researchers in China, are recognized as clinically useful drugs in Southeast Asia (FIG. 1).²³ Artesunate (7) has been licensed to Sanofi, which has launched an oral preparation, Arsumax®, in Western Africa and Rhone Poulenc Rorer has licensed artemether (6) only for use in severe malaria.
The lactol derivatives of artemisinin include the following:

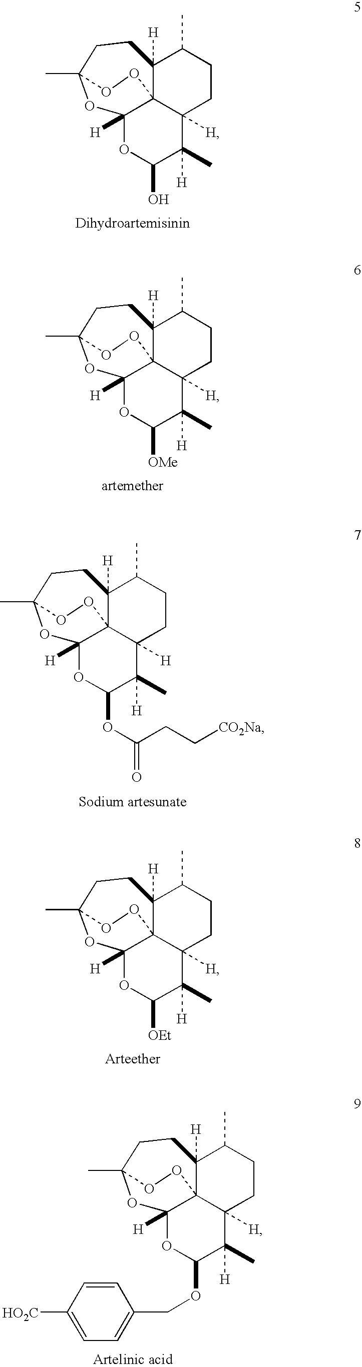

In the western world, the US Army has expended considerable effort towards clearance of arteether (8), currently available only in Asia, for human use.²⁴ The Walter Reed Army Institute of Research has also developed a more stable, water-soluble derivative related to artesunate (7), artelinic acid (9) (FIG. 2). A new malaria drug, Riamet®, already available in China, has been approved for sale by Novartis in Switzerland, its first Western market. This product, licensed from the Institute of Microbiology and Epidemiology in Beijing, combines artemether (6), with a synthetic substance, lumefantrine (10).

It is indicated for the treatment of P. falciparum malaria, including emergency treatment for travelers to regions where malaria is prevalent. While the development of second-generation artemisinin analogs is usefiul for the treatment of severe and complicated malaria, there are concerns about recent observations of fatal neurotoxicity of arteether (8) in animals. Studies have demonstrated that dihydroartemisinin (5), an in vitro metabolite of artemether (6), artesunate (7) and arteether (8), is probably the causative neurotoxic agent. To avoid metabolism to neurotoxic dihydroartemisinin (5), it has been suggested that future generations should avoid a hemiacetal type structure.^{25 26}

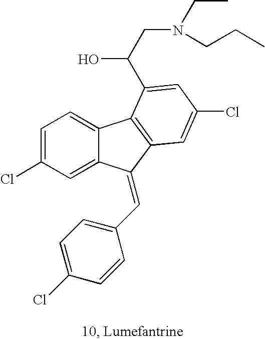

### SUMMARY OF THE INVENTION

This invention describes the synthesis, bioassay results and utility of new C-9, and C-10 substituted artemisinin derivatives with easily functionalizable groups attached to the artemisinin skeleton through carbon chain or heteroatoms. Described also is the demonstration of this class of compounds for their broad-spectrum anti-parasitic activity. Certain of these analogs in particular 12 and 14 possess noticeable cytotoxicity deliberately focused on treatment of cancerous diseases.

Shown below are C-9 substituted artemisinin and 10-deoxo artemisinin derivatives in monomeric (11) and dimeric (12) forms as well as C-10 substituted artemisinin monomers (13) and dimers (14).

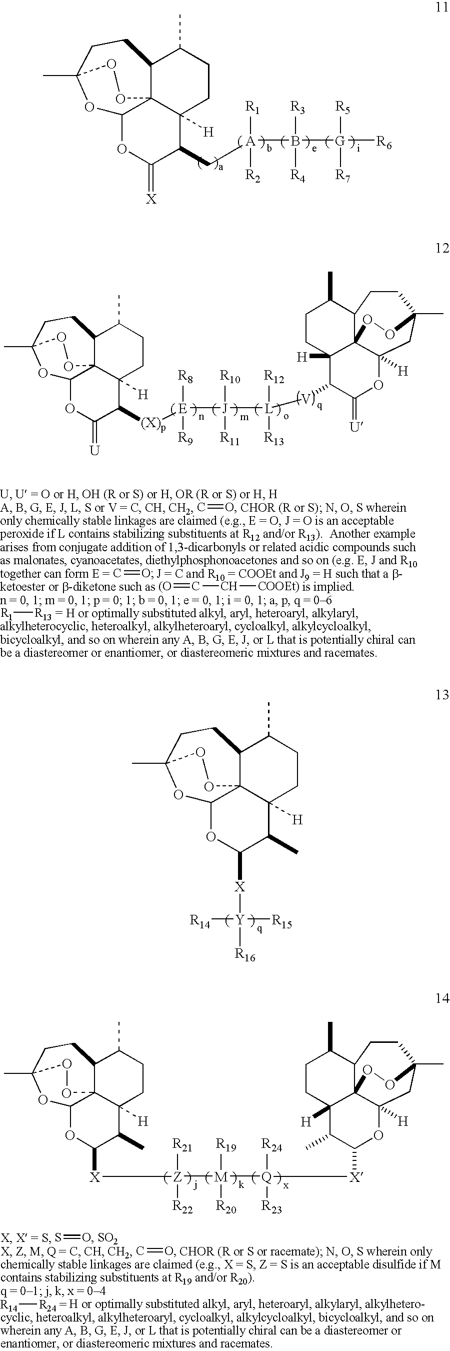

A versatile methodology for the construction of C-9 substituted analogs of artemisinin and synthesized new C-9 substituted artemisinin and C-9 substituted-10-deoxoartemisinin analogs (Scheme 1) for enhanced oral activity has been developed. These compounds contain either substituted alkyl or aryl groups or reactive functional groups attached to the C-9 position either through a carbon chain or through a heteroatom, which can be used to make libraries of artemisinin derivatives in a combinatorial or parallel synthesis fashion.

The facile interconversion of naturally occurring artemisinin 1 into artemisitene 16, also a natural product found in Artemisia annua, is of pivotal importance to the semisynthetic process for preparing the analogs described.

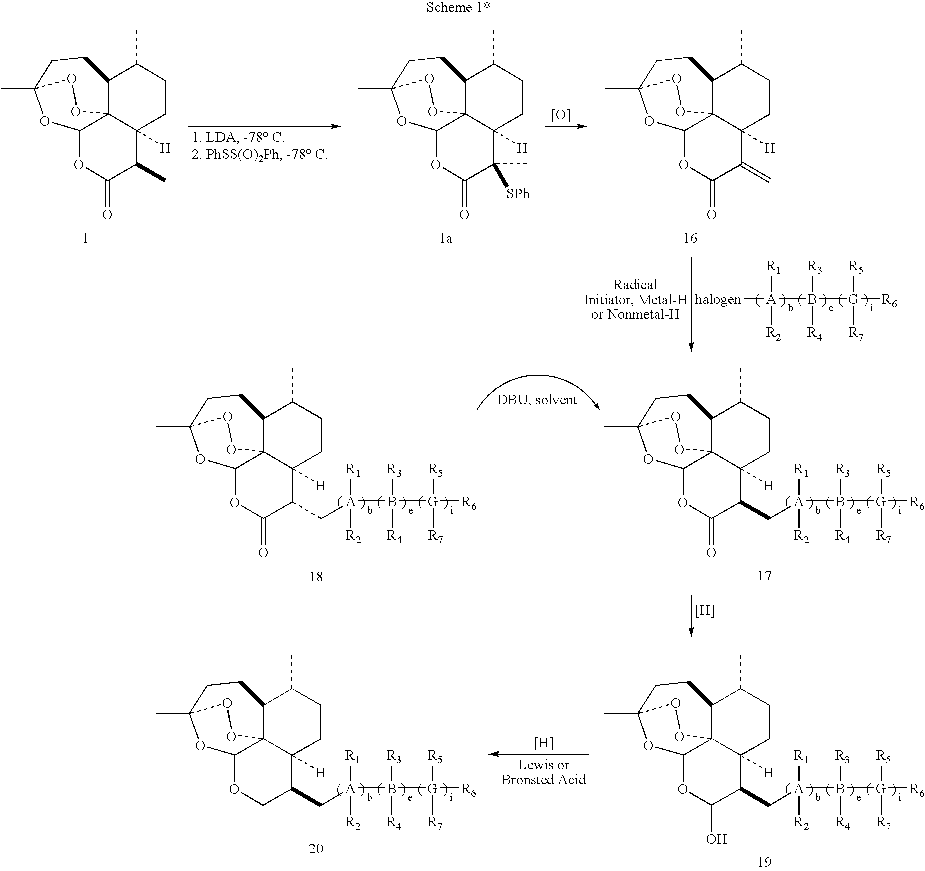

Trapping of the enolate of artemisinin, generated and kept during reaction at low temperature, with benzenethiosulfonic acid S-phenyl ester, leads to the a-thiophenyllactone intermediate 1a. In situ oxidation can be achieved by a variety of oxidants (e.g. meta-chloroperbenzoic acid; hydrogen peroxide; oxygen, tert-butylhydroperoxide, benzoyl peroxide, and so on), leading to an intermediate sulfone that undergoes spontaneous sigmatropic rearrangement, eliminating benzenesulfinic acid and thereby leading directly to artemisitene 16. This process can be conducted in a one-vessel operation, or in separate steps if so desired. Addition of carbon radicals to artemisitene is an enabling technology that allows for the construction of high potency derivatives from natural product. Conventionally, a primary halide can undergo hemolytic scission of the C—X bond leading to a carbon centered radical. These reactive intermediates readily undergo conjugate addition to artemisitene providing a mixture of C-9R or C-9S diastereomers than can be interconverted by base catalyzed equilibration with diazabicycloundecene or related amidinyl or guanidinyl containing moieties, or polymers. Normally, this radical chemistry is carried out using tributyltin hydride as a H atom donor (radical chain propagation) and aza-isobutyronitrile as an initiator. The hydrogen atom donor can be silanes (e.g. R₃SiH, R₂SiH.₂, etc.), polysilanes, polyoxasilanes (SiH₂OSiH₂O)n, silanes or polyoxasilanes in excess with catalytic R₃SnH; tris-trialkylsilylsilanes (TMS)₃SiH and other disilanes; Germanes such as R₃GeH and digermanes, and so on by analogy to Silicon; Phosphines such as PH₃, RPH₂, R₂PH, arsines, thiols and arylthiols (e.g. PhSH) comprise a short list of the enormous possible reagents. As for initiators, AIBN, benzoyl peroxide, but most notably, sunlight or photochemical generation of radicals is straightforward. Again, a long list of potential. initiators exists. Further, any of these reagents can exist in solvent, as solvent, on polymers or carriers, and so on such as to make the process as ecologically safe as possible. Organotins represent a possible source of pharmaceutical contamination, and ecological liability but can be replaced by innocuous silane reagents.

Once the radical addition has occurred and the material has been worked up, the isomeric mixture can be treated with DBU to equilibrate the alpha isomer 18 to the desired beta isomer 17. In some cases, both isomers are active. The use of DBU was peculiar to this transformation, weaker bases were less effective. Versions of DBU on polymeric support are available for process development. This process leads, after purification, to the lactone targets 17.

Removal of the lactone carbonyl leads to more active drugs, and can be accomplished either in one operation using a lewis acid in combination with a Hydride source. Such a combination is exemplified by BF3-etherate, NaBH₄ in methanol; or Et₃SiH and BF₃-etherate in dichloromethane. The reduction to lactol can be accomplished with typical reductants, but the peroxide is sensitive to this choice. Sodium borohydride can work if conducted carefully, as is the case for diisobutylaluminum hydride (DIBALH). Other reductants can be used, and combinations of reductant and Lewis acid can furnish pyran 20 directly from lactone 17.

Also synthesized were analogs of artemisinin having substitution at C-9 and C-10 through sulfur functionality containing side chain, which carries reactive functional groups such as amino, carboxyl etc, useful in the synthesis of a wide variety of artemisinin derivatives (Scheme 2-3). Other functionality for X and V in Scheme 2 for 21 can include oxygen and amines, peroxides. disulfides, hydrazines, hydrazones, etc.; but conjugate addition of sulfur occurs under mild conditions. Further oxidation of sulfur for X, such as R or S or racemic sulfones where X═SO are possible, as are the sulfones X═SO₂ In the case of the dimers 22, the same variables apply, but now X and V can either be symmetrical or mixed in nature, e.g. X could be S while V in the same material might be an O atom or even a beta-dicarbonyl system.

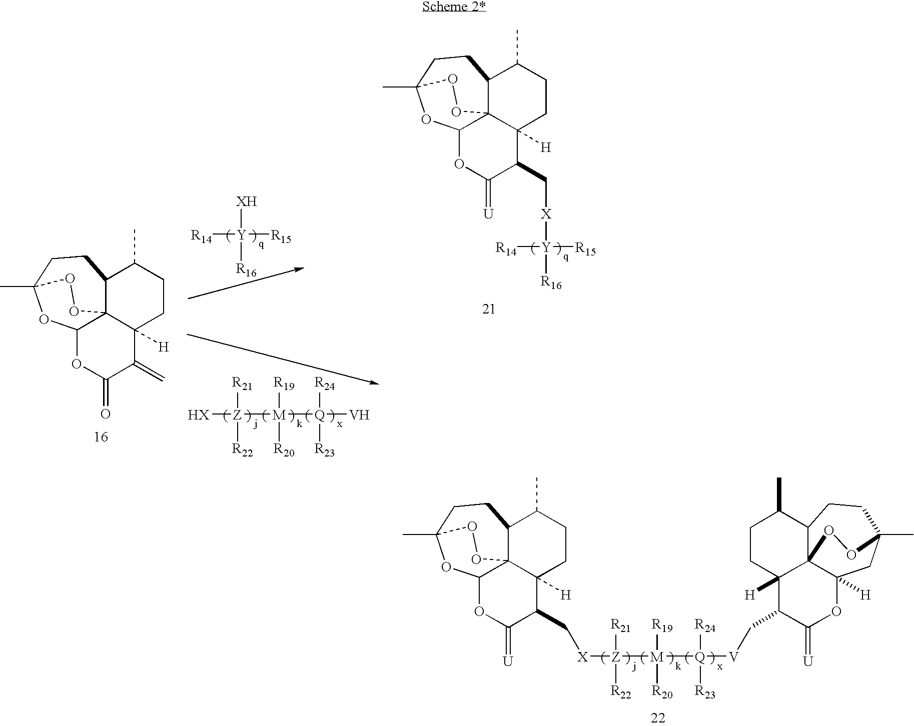

Many of these compounds have been evaluated in vitro and in vivo for antimalarial activity (Plasmodium falciparum and other plasmodia), for neurotoxicity, and against other parasitic organisms such as Leishania donovani, Babesia divergens, Toxoplasma gondii, Trypanosoma Cruzi (chaga's desease), Trypanosoma brucei (African sleeping sickness), Schistosoma japonica etc.

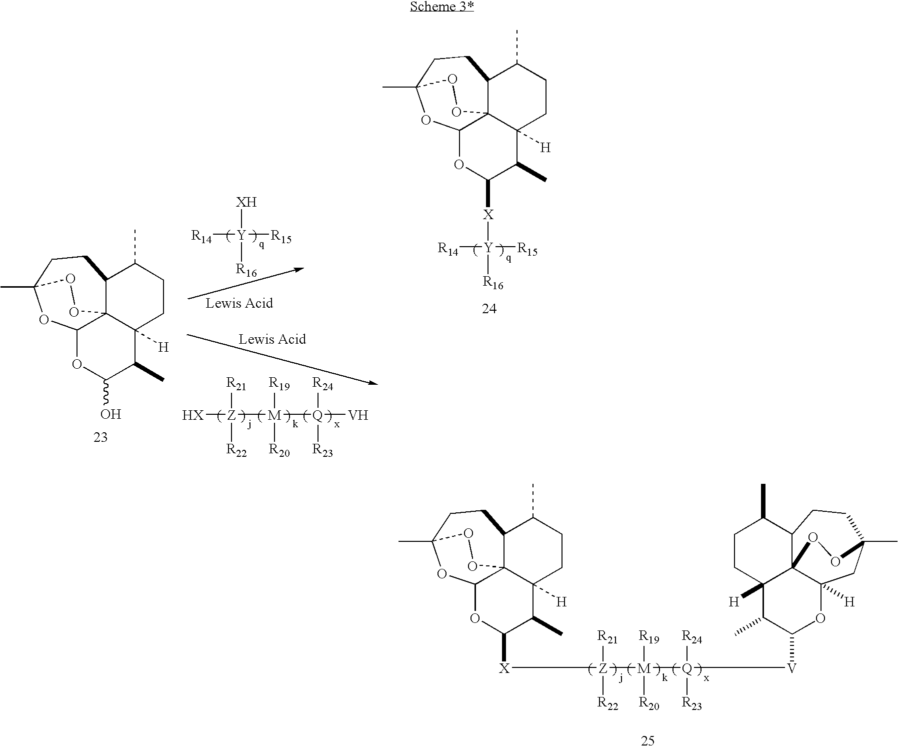

### DETAILED DESCRIPTION OF THE INVENTION

This patent describes the synthesis of C-9 and C-10 substituted artemisinin, and 9-substituted 10-deoxoartemisinins with substituted alkyl, aryl or other reactive functional groups attached to the main skeleton through either carbon chain or a heteroatom. The synthesis makes use of the natural product artemisinin or another naturally occurring compound, artemisitene as the starting material. These compounds have been shown to possess useful antiparasitic activity in vitro and in vivo, especially against malaria (e.g. Plasmodium falciparum) and Leishmania (L. donovani).

As used herein, the term “alkyl” refers to a straight or branched chain hydrocarbon having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkyl” group may containing one or more O, S, S(O), or S(O)₂ atoms. Examples of “⁴alkyl” as used herein include, but are not limited to, methyl, n-butyl, t-butyl, n-pentyl, isobutyl, and isopropyl, and the like.

As used herein, “cycloalkyl” refers to an alicyclic hydrocarbon group optionally possessing one or more degrees of unsaturation, having form three to twelve carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. “Cycloalkyl” includes by way of example cyclopropyl, cyclobutyl, cyclopentyl, cyclohexyl, cycloheptyl, or cyclooctyl, and the like.

As used herein, the term “heterocyclic” or the term “heterocyclyl” refers to a three to twelve-membered heterocyclic ring optionally possessing one or more degrees of unsaturation, containing one or more heteroatomic substitutions selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more of another “heterocyclic” ring(s) or cycloalkyl ring(s). Examples of “heterocyclic” include, but are not limited to, tetrahydrofuran, 1,4-dioxane, 1,3-dioxane, piperidine, pyrrolidine, morpholine, piperazine, and the like.

As used herein, the term “aryl” refers to a benzene ring or to an optionally substituted benzene ring system fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy optionally substituted by acyl, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of aryl include, but are not limited to, phenyl, 2-naphthyl, 1-naphthyl, 1-anthracenyl, and the like.

As used herein, the term “heteroaryl” refers to a five—to seven—membered aromatic ring, or to a polycyclic heterocyclic aromatic ring, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring systems, one or more of the rings may contain one or more heteroatoms. Examples of “heteroaryl” used herein are furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like.

As used herein, the term “alkoxy” refers to the group RₐO—, where Rₐ is alkyl.

As used herein, the term “alkylsulfonyl” refers to the group RₐSO₂—, where Rₐ is alkyl.

As used herein, the term “alkoxycarbonyl” refers to the group RₐOC(O)—, where Rₐ is alkyl.

As used herein, the term “acyloxy” refers to the group RₐC(O)O—, where Rₐ is alkyl, alkenyl, alkynyl, cycloalkyl, cycloalkenyl, or heterocyclyl.

As used herein, the term “optionally” means that the subsequently described event(s) may or may not occur, and includes both event(s) which occur and events that do not occur.

As used herein, the term “substituted” refers to substitution with the named substituent or substituents, multiple degrees of substitution being allowed unless otherwise stated.

As used herein, the terms “contain” or “containing” can refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

Whenever the terms “alkyl” or “aryl” or either of their prefix roots appear in a name of a substituent (e.g. arylalkoxyaryloxy) they shall be interpreted as including those limitations given above for “alkyl” and “aryl”. Alkyl or cycloalkyl substituents shall be recognized as being functionally equivalent to those having one or more degrees of unsaturation. Designated numbers of carbon atoms (e.g. C₁₋₁₀) shall refer independently to the number of carbon atoms in an alky, or cyclic alkyl moiety or to the alkyl portion of a larger substituent in which the term “alkyl” appears as its prefix root.

As used herein, the term “halogen” or “halo” shall include iodine, bromine, chlorine and fluorine.

As used herein, the term “acyl” refers to the group RₐC(O)—, where Rₐ is alkyl, alkenyl, alkynyl, cycloalkyl, cycloalkenyl, or heterocyclyl.

As used herein, the term “aroyl” refers to the group RₐC(O)—, where Rₐ is aryl.

As used herein, the term “heteroaroyl” refers to the group RₐC(O)—, where Rₐ is heteroaryl.

As used herein, the term “alkoxycarbonyl” refers to the group RₐOC(O)—, where Rₐ is alkyl.

As used herein, the term “acyloxy” refers to the group RₐC(O)O—, where Rₐ is alkyl, alkenyl, alkynyl, cycloalkyl, cycloalkenyl, or heterocyclyl.

As used herein, the term “alkoxycarbonyl” refers to the group RₐOC(O)—, where Rₐ is alkyl, alkenyl, alkynyl, cycloalkyl, cycloalkenyl, or heterocyclyl.

As used herein, the term “carboxy” shall refer to the substituent —COOH.

As used herein, the term “cyano” shall refer to the substituent —CN.

As used herein, the term “aminosulfonyl” shall refer to the substituent —SO₂NH₂.

As used herein, the term “carbamoyl” shall refer to the substituent —C(O)NH₂.

As used herein, the term “sulfanyl” shall refer to the substituent —S—.

As used herein, the term “sulfenyl” shall refer to the substituent —S(O)—.

As used herein, the term “sulfonyl” shall refer to the substituent —S(O)2—.

Any racemates can be split in a manner know per se, for example, after conversion of the optical antipodes into diastereoisomer, for example, by reaction with optically active acids or bases.

The pharmacologically acceptable compounds of the present invention can be used, for example, for the manufacture of pharmaceutical compositions which contain an effective amount of the active substance together or in admixture with inorganic or organic, solid or liquid, pharmaceutically acceptable carriers.

The pharmaceutical compositions according to the invention are those which are suitable for enteral, such as oral, administration and for parenteral, such as subcutaneous, administration to warm-blooded animals, especially humans, and which contain the pharmacologically active substance on its own or together with a pharmaceutically acceptable carrier. The dosage of the active substance depends on the species of warm-blooded animal and on the age and individual condition the illness to be treated and also on the mode of administration.

The pharmaceutical compositions of the present invention are manufactured in a manner know per se, for example, by means of conventional mixing, granulating, confectioning, dissolving or lyophilizing process. Pharmaceutical compositions for oral use can be obtained by combining the active substance with one or more solid carriers, if desired, granulating a resulting mixture and processing the mixture or granulate, if desired or necessary after the addition of suitable adjuncts, to form tablets or dragee cores. In doing so, they can also be incorporated into plastics carriers which release the active substances or allow them to diffuse in controlled amounts.

Suitable carriers are especially fillers such as guars, for example, lactose, saccharose, mannitol or sorbitol, cellulose preparations and/or calcium phosphates, for example, tricalcium phosphate or calcium hydrogen phosphate, also binders such as starches, for example, corn, wheat, rice or potato starch, gelatine, tragacanth, methylcellulose, hydroxypropylmethylcellulose, sodium carboxymethycellulose and/or polyvinylpyrrolidone, and/or, if desired, disintegrators such as the above-mentioned starches, also carboxymethyl starch, cross-linked polyvinylpyrrolidone, agar, alginic acid or a salt thereof such as sodium alginate. Adjuncts are especially flow-regulating and lubricating agents, for example, silica, talc, stearic acid or salts thereof such as magnesium or calcium stearate, and/or polyethylene glycol. Dragee cores are provided with suitable coatings that are, if desired, resistant to gastric juice, there being used, inter alia, concentrated sugar solutions which optionally contain gum arabic, talc, polyvinylpyrrolidone, polyethylene glycol and/or titanium dioxide, lacquer solutions in suitable organic solvents or solvent mixtures or, for the manufacture of coatings that are resistant to gastric juice, solutions of suitable cellulose preparations such as acetylcellulose phthalate or hydroxypropylmethylcellulose phthalate. Coloring substances or pigments can be added to the tablets or dragee coatings, for example for the purpose of identification or for indicating different doses of active substance.

Other orally administrable pharmaceutical compositions are dry-filled capsules made of gelatin and also soft, sealed capsules made of gelatin and a plasticizer such as glycerol or sorbitol. The dry-filled capsules may contain the active ingredient in the form of granulate, for example, in admixture with fillers such as corn starch, binders and/or glidants such as talc or magnesium stearate and optionally stabilizers. In soft capsules, the active ingredient is preferably dissolved or suspended in suitable liquids or wax-like substances such as fatty oils, paraffin oil or polyethylene glycols, it being possible also for stabilizers to be added.

Other form s of oral administration are, for example, syrups prepared in a customary manner that contain the active ingredient in, for example, suspended form and in a concentration of approximately 5% to 20%, and preferably approximately 10%, or in similar concentration that provides a suitable single dose when administered, for example, in measures of 5 or 10 ml. Also suitable are, for example, powdered or liquid concentrates for preparing shakes, for example, in milk. Such concentrates can also be packed in single-dose quantities.

Particularly suitable dosage forms for parenteral administration are sterile aqueous solutions of an active ingredient in water-soluble form, for example, a water-soluble salt, or sterile aqueous injection suspensions which contain substances increasing the viscosity, for example, sodium, carboxymethyl cellulose, sorbitol and/or dextran, and optionally stabilizers. In addition, the active ingredient, with or without adjuvants, can also be in lyophilized form and brought into solution prior to parenteral administration by the addition by the addition of suitable solvents.

The actual dosage amount administered can be determined by physical and physiological factors such as body weight, severity of condition, and idiopathy of the patient. With these considerations in mind, the dosage of any of the presently claimed compounds for a particular subject and or course of treatment can readily be determined.

The term “pharmacologically effective amount” or shall mean that amount of a drug or pharmaceutical agent that will elicit the biological or medical response of a tissue, animal or human that is being sought by a researcher or clinician. This amount can be a therapeutically effective amount. The term “therapeutically effective amount” shall mean that amount of a drug or pharmaceutical agent that will elicit the therapeutic response of an animal or human that is being sought.

The term “treatment” or “treating” as used herein, refers to the full spectrum of treatments for a given disorder from which the patient is suffering, including alleviation of one, most of all symptoms resulting from that disorder, to an outright cure for the particular disorder or prevention of the onset of the disorder.

Synthesis of Compounds

The synthesis of new artemisinin derivatives, which are expected to have improved bioavailability, is presented in Scheme 4. As could be seen, a selenium-free route was employed in the synthesis of artemisitene, which was then reacted with the appropriate alkyl or arylalkyl bromide synthons.

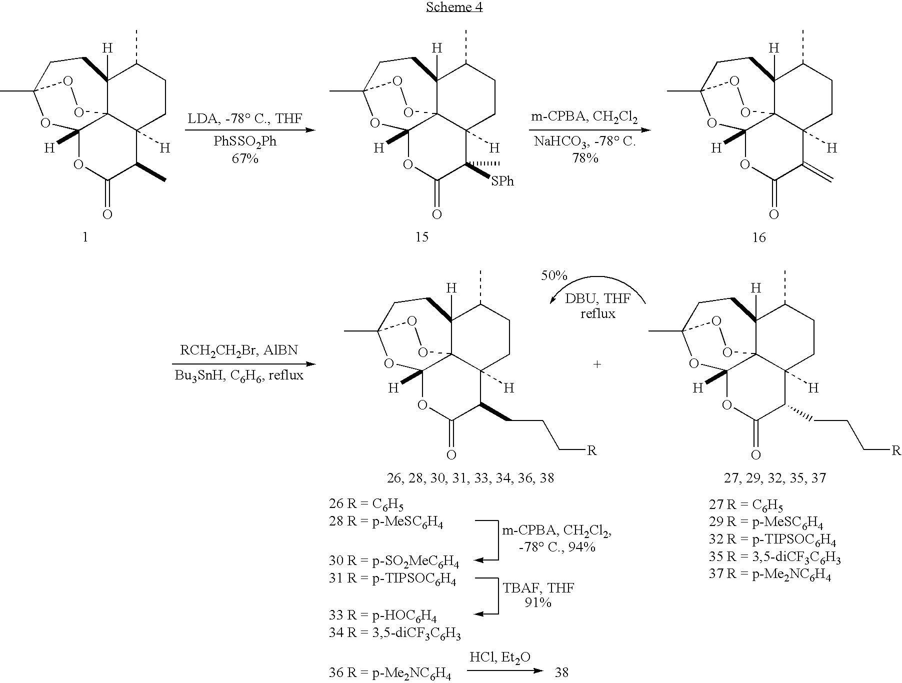

For radical-induced Michael addition of artemisitene, a dilute solution of tributyltin hydride was added slowly over 8h, via syringe pump, to a refluxing solution of artemisitene, AIBN, and the corresponding bromide. The solvent was concentrated to 25 mL, stirred with a saturated solution of KF for 10h, filtered, and concentrated. The products were separated by column chromatography to afford the α- and β-epimers. Yield of the β-isomer ranged from 14-32% and that of α-isomer from 30-43%. The 9α-substituted analogues were converted to the 9β-congeners by refluxing with DBU in THF for 12H.

As shown in Scheme 5, the bromide synthons used (41a-41c) were synthesized from the corresponding acids first by reduction to alcohols followed by bromination using PPh₃/CBr₄ (for 41a) or PPh₃/Br₂ (for 41b and 41c).

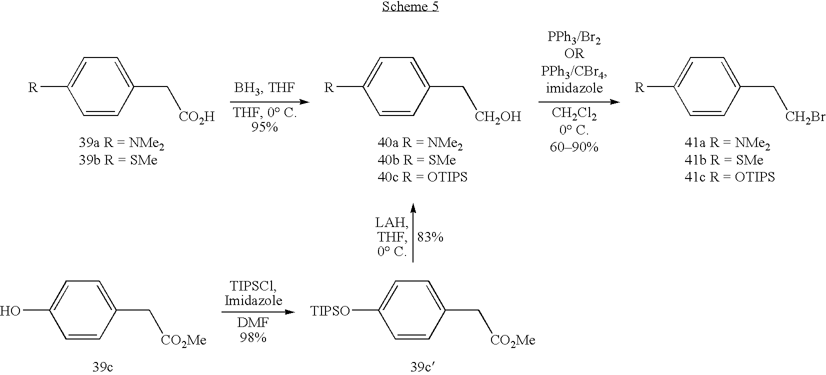

The 10-deoxo derivative 43 was synthesized from compound 36 in 35% yield using a two step reaction sequence involving two reductions, first affording the lactol (42) in 82% yield, followed by a second reduction with triethylsilane and boron trifluoride etherate (Scheme 6). Due to the potential instability of the peroxide group in the presence of the amino function, 27 the N, N-dimethylamino derivatives 36 and 43 were converted to the corresponding hydrochloride salts 38 and 43a respectively, by treatment with equimolar amounts of HCl in ether. The formation of hydrochloride salts was evidenced by the shift of ¹H NMR signals corresponding to the NMe₂ groups from δ2.9 to 3.14 and that of aromatic doublets from 6.69 and 7.04 to 7.28 and 7.65, respectively. The conversion of sulfide (28) to sulfone (30) was accomplished in 94% yield by treatment with mCPBA at −78° C. in CH₂Cl₂. Similarly, the silyl group in 31 could be removed conveniently using tetrabutyl ammonium fluoride in THF, at room temperature to afford 91% of 33.

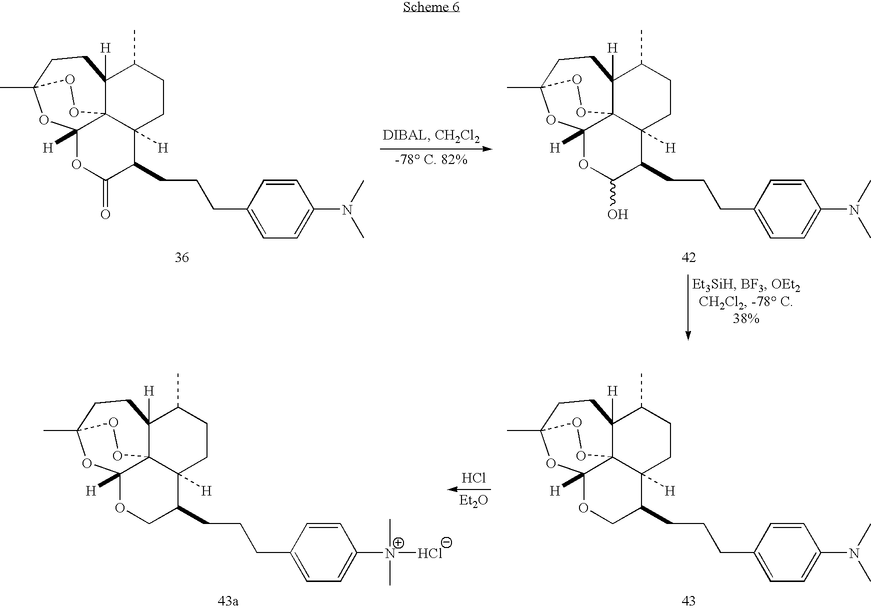

Radical induced Michael addition strategy was also employed in the synthesis of various artemisinin derivatives bearing reactive functional groups like, NH₂, CN, aldehyde equivalent (1,3-dioxalane) or OH (in the form of OTHP) at the C-9 position (Scheme 7). To introduce amino functionality, the amino group of commercially available bromoethylamine hydrochloride was first protected using Boc carbonate in about 70% yield and then used in the Michael addition reaction as described previously. The a isomer (44) was converted to the β form (45) by refluxing with DBU in THF for 10 h. The β form was converted to the 10-deoxo derivative first by reducing to the lactol (46), followed by treatment with Et₃SiH/BF₃.OEt₂ to afford 47. The utility of the amino functionality for the synthesis of various analogs was demonstrated by the preparation of the dansyl derivative 48 useful as fluorescent marker to identify the protein targets where this class of compounds acts.

Similarly cyano, aldehyde and alcohol functionalities were attached to the artemisinin skeleton by radical induced Michael addition strategy using commercially available bromoacetonitrile (49), 2-bromoethyl-1,3-dioxalane (50), and 2.(2-bromoethoxy)tetrahydro-2H-pyran (51) as bromide synthons to afford 52, 53 and 54 respectively (Scheme 7).

Also explored was the possibility of using thiol functionality as an anchoring point to artemisitene by a similar Michael addition approach. This could be exemplified by the synthesis of artemisinin-cysteine adduct (55, Scheme 8) which offers a good opportunity to use suitably functionalized thiols like 56 for derivatization of artemisinin.

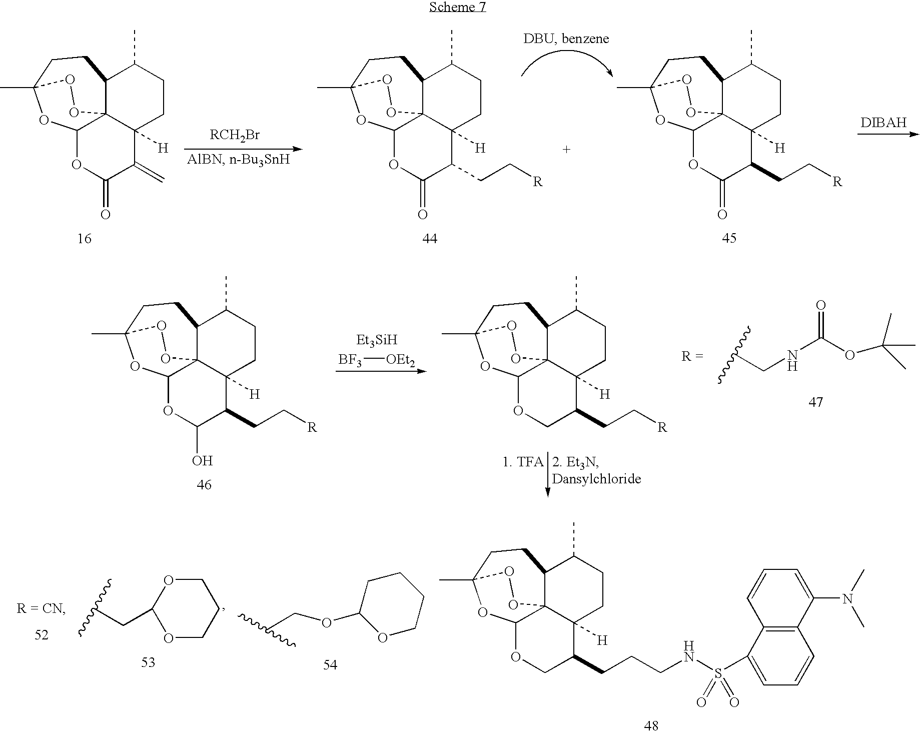

Another approach, which was pursued in order to attach various functional groups to artemisinin, was the preparation of thioether derivatives at C-10. This can be exemplified by the synthesis of the cysteine adduct 58 (Scheme 9) by treating dihydroartemisinin with cysteine in presence of BF₃.OEt₂. Similar strategy can be used to prepare dimeric artemisinin derivatives of the type 57.

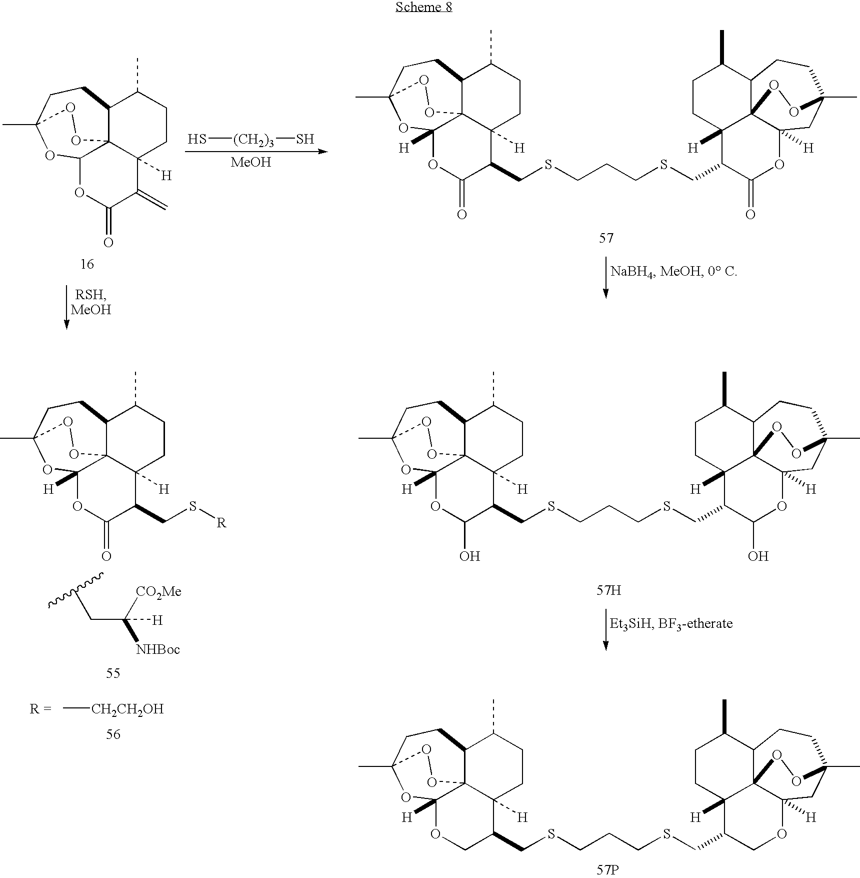

This work demonstrates that artemisitene can be used as the precursor to produce artemisinin derivatives with new functionality at C-9. Also demonstrated was the possibility of synthesizing various C-1 thioether derivatives of artemisinin from dihydroartemisinin. The routes described here are reasonably simple pathways to synthesize libraries of C-9β and C-10 substituted artemisinin analogs. Since a primary consideration is oral potency and duration of action, variation in the side-chain is important to achieve fine-tuning of the pharmacokinetic profile. As shown in FIG. 2 and 3, a plethora of alkyl, aryl and heteroaryl substituents can be incorporated into the artemisinin skeleton to achieve this goal. 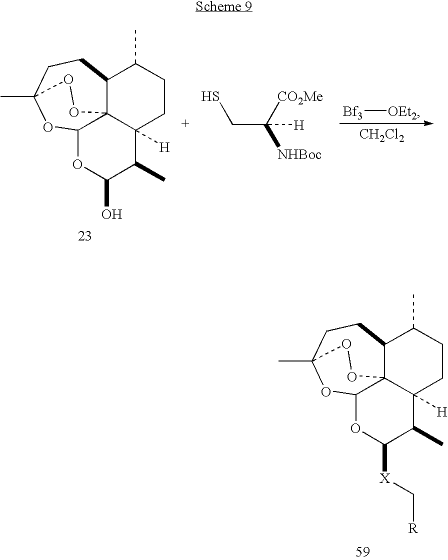
Biological Activity

The WHO/TDR in vitro antimalarial screen was conducted using modifications of the procedures of Desjardins et al., to assess the intrinsic activity of the new analogs 36-41 relative to known controls such as quinine (2), chloroquine (3), mefloquine (4), and sodium artesunate (7). This method can provide quantitative measurements of the antimalarial activity, based on the inhibition of uptake of radiolabeled nucleic acid precursor by the parasite during short-term cultures in microtritation plates.³⁶ Pure samples were tested for antimalarial activity in vitro in parasitized human red blood cells (RBC) against four Plasmodium falciparum clones: mefloquine-resistant, chloroquine-sensitive Sierra Leone (1)6), chloroquine-sensitive NF54, chloroquine-resistant K1 and chloroquine-resistant, mefloquine-sensitive Indochina (W2). All test compounds are solubilized in DMSO and diluted 400 fold (to rule out a DMSO effect) in culture medium with plasma for a starting concentration of at least 12,500 ng/mL. Drugs are subsequently diluted five fold using the Cetus Pro/Pette system utilizing a range of concentrations from 0.8 ng/mL to 12,500 ng/mL. Fifty percent inhibitory concentrations are reported in ng/mL.

The D6 African P. falciparum clone is sensitive to chloroquine, pyrimethamine and sulfadoxine but resistant to mefloquine. The W2 Indochina P. falciparum clone is resistant to chloroquine, pyrimethamine and sulfadoxine but sensitive to mefloquine. The K1 P. falciparum is a resistant strain, where NF54 P. falciparum is a sensitive strain.

TABLE 1
In vitro data (P. falciparum)
P. falciparum (IC₅₀)

| Com- | | D6 | W2 | K1 | NF54 |
|---|---|---|---|---|---|
| pound | MW | ng/mL | ng/mL | ng/mL | ng/mL |
| Chloro- | 319.88 | 7.58 | 118.04 | 79.01 | 7.36 |
| quine | | | | | |
| Meflo- | 442.28 | 45.55 | 34.06 | 20.34 | 35.38 |
| quine | | | | | |
| Quinine | 342.42 | 159.61 | 149.88 | 99.60 | 9.08 |
| 11 | 405.93 | 1.9 (1.9) | 3.0 (2.2) | NT | NT |
| 36 | 405.93 | 7.55 | 21.60 | 10.07 | 16.27 (40.19) |
| 37 | 390.465 | 8.28 | 26.83 | 11.56 | 41.76 (27.33) |
| 38 | 439.49 | 8.48 | 30.32 | 12.00 | 35.60 (30.76) |
| 39 | 439.49 | 28.13 | 58.01 | 39.55 | 101.52 (48.78) |
| 40 | 508.49 | NT | 7.11 | NT | NT |
| 41 | 408.48 | NT | 6.70 | NT | NT |

*11 = p-Cl 36 (not m-Cl).

The results of the in vivo Peter's 4-day antimalarial testing³⁷ of our compounds revealed that the new analogs have good oral activities (Table 2). Factors including stability, synthesis cost, and activity, led to the selection of compounds 39 (the most potent) and 36 (the most economical) for synthesis of 5 gram scale-up for pharmacokinetic analysis (PK).

In the “4day test” for each compound under test, two batches of five male random-bred Swiss albino mice weighing 18-22 g are incubated intravenously with 2×10⁶ RBC parasitized with P. berghei N. Animals are then given a fixed dose of 30 mg(Kg once a day for four consecutive days. Compounds are first dissolved in DMSO (due to lower water solubility) and subsequently an aqueous dilution is prepared for use and administered subcutaneously to one group and orally to a second group.

TABLE 2
In vivo data (P. berghei N)

| P. berghei N S.C. | | | P. berghei N P.O. | |
|---|---|---|---|---|
| Compound | ED₅₀ | ED₉₀ | ED₅₀ | ED₉₀ |
| Chloroquine | 1.5 | 3 | 1.7 | 3.1 |
| Mefloquine | 1.5 | 3.8 | 1.1 | 2.3 |
| Quinine | 25 | 190 | 71 | 118 |
| Sodium artesunate | 1.5 | 6.3 | 2.4 | 13.0 |
| 11 | 0.7 | 1.2 | 1.0 | 2.1 |
| 36 | 0.5 (0.1-1.0) | 0.9 (0.3-1.8) | 2.0 (0.9-3.5) | 8.3 (3.5-14.0) |
| 37 | 0.46 (0.19-1.1) | 0.85 (0.36-2.3) | 0.9 (0.38-1.9) | 3.4 (1.45-7.5) |
| 38 | 0.4 (0.2-1.2) | 0.75 (0.4-2.1) | 1.25 (0.5-2.3) | 4.4 (1.3-6.2) |
| 39 | 0.44 (0.25-0.62) | 0.85 (0.5-1.2) | 0.63 (0.32-1.25) | 2.1 (1.1-4.1) |
| 40 | <10 | <10 | <10 | <10 |
| 41 | <10 | <10 | <10 | <10 |

TABLE 3
In vivo data for compound 35 (P. yoelli ssp. NS and P. berghei N)

| | | | ED₅₀ | ED₉₀ | % |
|---|---|---|---|---|---|
| Compound | Strain | Route | mg/kg × 4 | mg/kg × 4 | Suppression |
| 35 | N | PO | >10 | >10 | 6.5 |
| 35 | NS | PO | NA 10 | NA 10 | 0.3 |
| Sodium artesunate | N | PO | <10 | >10 | 77.2 |
| Sodium artesunate | NS | PO | <10 | >10 | 56.5 |

TABLE 4
In vivo data (P. yoelli ssp. NS)

P. yoelli ssp. NS (S.C.)

| Compound No | ED50 | ED90 |
|---|---|---|
| Chloroquine | 1.6 | 28 |
| Mefloquine | 2.3 | NT |
| Quinine | 128 | NT |
| Sodium artesunate | 5.3 | 23.3 |
| 11 | 0.6 | 1.0 |
| 36 | 0.60 (0.4-1.0) | 1.00 (0.8-1.8) |
| 37 | 0.58 (0.45-1.0) | 1.00 (0.8-1.8) |
| 38 | 0.60 (0.3-0.9) | 1.00 (0.5-1.5) |
| 39 | 0.67 (0.5-0.88) | 1.25 (0.95-1.6) |

The parasitemia is determined on the day following the last treatment—day 4 to determine qualitatively the presence and degree of activity at the screening dose.

Preliminary data for the PK results of compounds 39 and 36 have been disclosed. The objective of this study was to evaluate the key pharmacokinetic parameters of the artemisinin derivatives 39 and 36 in rats after intravenous administration, and to then compare these data to dihydroartemisinin, which is being used as a reference compound. The absolute oral bioavailability of 39 and 36 was also evaluated. Due to their very low aqueous solubility, the absolute oral bioavailability of each compound was low. The low value is rather a function of the high oral dose (˜120 mg/kg), but even with these high doses, the plasma concentrations are still relatively low.

The ED90 data indicated that doses <10 mg/kg are effective in the mouse models for these compounds, so in the future, there are going to consider lower oral doses. The reason for the use of high oral dose is that the LC/MS assay has different intrinsic sensitivities for the different series of the Malperox compounds. Also, in order to compare all compounds from the different series at the same oral molar dose (dictated by the compound, which has the highest LOQ in the assay), it was necessary for them to have gone with this dose.

Compounds 39 and 36 have reasonably low plasma LOQ of about 10 ng/mL, so it will be tested with lower oral doses in the coming future. If we assume that it is only the fraction of drug in solution that is absorbed, then the oral bioavailabilities may well increase with lower doses. However, these two compounds still have very low aqueous solubilities (pH 6.5, phosphate buffer) of approximate 0.1 microgram per mL—hence, this remains a key issue.

In terms of the IV PK data (e.g. FIG. 4), these two compounds have the clearance values of 20-30 ml/min/kg for 39 and 36. The half-life for 39 is also reasonably good at 73 min, with the shorter value for 36 being an issue. Solubility limitations meant that 36 could only be dosed IV at approximate 2 mg/kg (compared with the approximate 12 mg/kg dose for 39). It is possible that the shorter measured half life for 36 was a function of lower plasma concentrations, which have reflected part of the distribution phase rather than a true elimination phase. The very low aqueous solubility of 39 and 36 is a significant limitation that can be addressed by increasing the polarity of the compounds (e.g. FIG. 5).

[image]

The Thad Cochran National Center for Natural Products Research (University of Mississippi) in vitro antimalarial screen was conducted using an enzyme assay for detection of P. falciparum (see procedure below). This assay is based on the observation that lactate dehydrogenase (LDH) of P. falciparum is distinguishable from host LDH activity. P. falciparum LDH rapidly uses 3-acetyl pyridine nicotinamide adenine dinucleotide (APAD) as a coenzyme in the biotransformation of pyruvate from lactate.³⁸ The same process is carried out in human red blood cells at a very slow rate.

The “Antimalarial parasite LDH assay” is currently being conducted according the following procedure: prepare a suspension of RBC with a 2% parasitemia and 2% hematocrit in malaria complete medium. Dispense 200 μl aliquots of this suspension into each well of a 96 well microtiter plate. Add 10 μl volumes of the drugs to be tested in duplicate to the appropriate wells. Place the plates into humidified chamber and flush the cultures with a gas mixture consisting of 90% N₂, 5% O₂, and 5% CO₂. Transfer the chamber containing the plates to an incubator, and incubate for 48 hours at 37° C. After 48 hours add 100 μl aliquots of the Malstat™ reagent to each well of a new 96-well microtiter plate Resuspend the cultures from the assay plate by mixing each well up and down several times. Remove 20 μl from each well of the resuspend culture and add to the plate containing the Malstat™ reagent. Incubate the plates at room temperature for 30 minutes. After 30 minutes, add to each well 20 μl of the 1:1 mixture of the NBT/PES solution (2 mg/ml and 0.1 mg/ml, respectively). Incubate the plates in the dark for 1 hour. Add 100 μl of 5% acetic acid solution to stop the reaction. The plate is then read at an endpoint of approximately 650 nm. The dose response curves are generated using Microsoft Excel.

TABLE 6
Antiprotozoal screening assays.

| | | | | | Cytotoxicity |
|---|---|---|---|---|---|
| P. falciparum (IC₅₀) | | | | | (Vero) |
| Compound No | D6 ng/mL | S. I.^{a} | W2 ng/mL | S. I.^{a} | TC₅₀ ng/mL |
| 35 | 90 | >53.0 | <19.5 | >244.0 | NC |
| 36 | 7.6 | >63.0 | 8.0 | >60 | NC^{b} |
| 37 | 11.0 | >43.0 | 12.0 | >40 | NC |
| 38 | 8.7 | >55.0 | 11.0 | >43 | NC |
| 39 | 12 | >40 | 12 | >40 | NC |
| 40 | 12 | >40 | 9.5 | >50 | NC |
| 41 | 3.8 | >125 | 2.3 | >207 | NC |

^{a}Selectivity Index (S. I.) = IC₅₀ (Vero cells)/IC₅₀ (P. falciparum)
^{b}NC = No cytotoxity

In vitro antimalarial and antileismania assay

Antimalarial activity of the analogues was determined in vitro on chloroquine sensitive (D)6, Sierra Leone) and resistant (W2, IndoChina) strains of Plasmodium falciparum. The 96 well microplate assay is based on evaluation of the effect of the compounds on the growth of asynchronous cultures of P. falciparum, determined by the assay of parasite lactate dehydrogenase (pLDH) activity.²⁸ The appropriate dilutions of the compounds were prepared in DMSO or RPMI-1640 medium and added to the cultures of P. falciparum (2% hematocrit, 2% parasitemia) set up in clear flat bottomed 96 well plates. The plates were placed into the humidified chamber and flushed with a gas mixture of 90% N₂, 5% CO₂ & 5% O₂. The cultures were incubated at 37° C. for 48 hours. Growth of the parasite in each well was determined by pLDH assay using Malstat® reagent. The medium and RBC controls were also set-up in each plate. The standard antimalarial agents, chloroquine and artemisinin, were used as the positive controls while DMSO was tested as the negative control.

Antileishmanial activity of the compounds was tested on a transgenic cell line of Leishmania donovani promastigotes expressing firefly luciferase.^{29a} In a 96 well microplate assay, compounds with appropriate dilution were added to the leishmania promastigotes culture (2×10⁶ cell/mL). The plates were incubated at 26° C. for 72 hours and growth of leishmania promastigotes was determined by luciferase assay with Steady Glo® reagent (Promega, USA). Pentamidine and Amphotericin B were used as standard antileishmanial agents. The analogues were simultaneously tested for cytotoxicity on VERO cells (monkey kidney fibroblast) by Neutral Red assay.^{29b} IC₅₀ values for the compounds were computed from the growth inhibition curve.

Biological Testing Results and Discussion

Antimalarial activity of the new analogues synthesized is presented in Table 5. These compounds were designed and synthesized to improve oral bioavailability and to understand the effect of C-9α/C-9β substitution on the antimalarial as well as leishmanicidal activities. The analogues exhibited promising in vitro activity against both P. falciparum and leishmania promastigotes (L. donovani), thus offering opportunity to develop broad spectrum antiparasitic agents.

In order to determine the various factors affecting the antileishmanial activity, a series of artemisinin analogs having diverse structural features were selected and tested for their antileishmania activity. To our surprise, most of the artemisinin analogues tested were notably active against leishmania. The activity of these molecules against leishmania is presented in Tables 1-Table 4. In general, these molecules can be classified into six different structural classes depending upon the nature and sites of derivatization ^{30, 31, 32} 1) alkyl or arylalkyl groups at the C-3 position, 2) alkyl or arylalkyl groups at C-9, 3) analogues with substitution at both C-3 and C-9 positions, 4) 10-deoxo compounds, 5) lactams, and 6) derivatives that lack one or the other of the artemisinin ring systems.

TABLE 1
Antileishmanial activity of artemisinin analogues^{a}.

| No | R₁ | R₂ | R₃ | IC₅₀ (μM)^{a} |
|---|---|---|---|---|
| 1 | CH₃ | CH₃ | H | 124.0 |
| | (artemisinin) | | | |
| 60 | CH₃ | H | H | 242.3 |
| 61 | CH₃ | Et | H | 60.7 |
| 62 | CH₃ | CH₃ | CH₃ | 101.2 |
| 63 | CH₃ | ⁿPr | H | 16.8 |
| 64 | CH₃ | —CH₂—CH═CH₂ | H | 17.8 |
| 65 | CH₃ | ⁿBu | H | 12.3 |
| 66 | CH₃ | —(CH₂)₃CH(CH₃)₂ | H | 8.5 |
| 67 | CH₃ | —(CH₂)₃CH(CH₃)₂ | H | 79.3 |
| 52 | CH₃ | —(CH₂)₂CN | H | 38.9 |
| 68 | CH₃ | —CH₂Ph | H | 15.3 |
| 69 | CH₃ | —(CH₂)₂Ph | H | 40.3 |
| 26 | CH₃ | —(CH₂)₃Ph | H | 11.6 |
| 27 | CH₃ | H | —(CH₂)₃Ph | 45.3 |
| 70 | CH₃ | —(CH₂)₄Ph | H | 7.5 |
| 71 | CH₃ | —(CH₂)₃C₆H₄(m-F) | H | 3.7 |
| 72 | CH₃ | —(CH₂)₃C₆H₄(p-Cl) | H | 11.9 |
| 73 | CH₃ | —(CH₂)₃C₆H₄(pOMe)) | H | 12.0 |
| 28 | CH₃ | —(CH₂)₃C₆H₄(p-SMe) | H | 1.4 |
| 30 | CH₃ | —(CH₂)₃C₆H₄(p-SO₂Me) | H | 53.8 |
| 33 | CH₃ | —(CH₂)₃C₆H₄(p-OH) | H | 44.7 |
| 34 | CH₃ | —(CH₂)₃C₆H₄(3,5-diCF₃) | H | 14.4 |
| 35 | CH₃ | H | —(CH₂)₃C₆H₄(3,5-diCF₃) | NA^{b} |
| 74 | CH₃ | —(CH₂)₃C₆H₄(3,5-diF) | H | 49.7 |
| 38 | CH₃ | —(CH₂)₃C₆H₄(p-NMe₂.HCl) | H | 1.9 |
| 37 | CH₃ | H | —(CH₂)₃C₆H₄(p-NMe₂.HCl) | 88.3 |
| 75 | Et | H | H | 177.1 |
| 76 | ⁿPr | H | H | 40.5 |
| 77 | ⁿBu | H | H | 257.7 |
| 78 | —(CH₂)₂CO₂Et | H | H | 31.0 |
| 79 | —(CH₂)₂Ph | H | H | 12.6 |
| 80 | (CH₂)₃C₆H₄-pCl | H | H | 11.1 |
| 81 | Et | ⁿBu | H | 35.5 |
| 82 | —(CH₂)₄Ph | ⁿBu | H | 4.5 |

TABLE 2

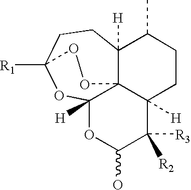

| | | | | |
|---|---|---|---|---|
| 83 | CH₃ | H | H | 44.4 |
| 84 | CH₃ | CH₃ | H | 70.3 |
| 85^{c} | CH₃ | CH₃ | H | 44.8 |
| 86^{d} | CH₃ | ⁿBu | H | 382.9 |
| 87 | CH₃ | —(CH₂)₃CF₃ | H | 26.3 |
| 88^{d} | CH₃ | —(CH₂)₃C₆H₄(p-CF₃) | H | 2.9 |
| 89^{d} | CH₃ | —(CH₂)₃C₆H₄(m-Cl) | H | 16.6 |
| 90^{d} | CH₃ | —(CH₂)₃C₆H₄(p-Cl) | H | 4.7 |
| 91 | CH₃ | —(CH₂)₃C₆H₄(3,5-diF) | H | 33.0 |
| 92^{b} | CH₃ | —(CH₂)₃C₆H₄(3,5-diCF₃) | H | NA |

^{b}Not active up to 125 μg/ml.
^{c}as arteether
^{d}These compounds showed cytotoxicity in the concentration of 2000-2700 μg/ml while all the other compounds did not exhibit observable cytotoxicity as measured by the procedure described in the text.

TABLE 3

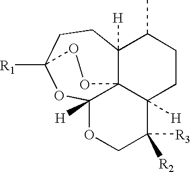

| | | | | |
|---|---|---|---|---|
| 93 | CH₃ | H | H | 49.2 |
| 94 | CH₃ | CH₃ | H | 46.6 |
| 95 | CH₃ | —(CH₂)₃C₆H₄(pCl) | H | 4.9 |
| 96 | CH₃ | —(CH₂)₃C₆H₄(mCl) | H | 3.7 |
| 97 | CH₃ | —(CH₂)₃C₆H₄(3,4-diCl) | H | 2.3 |
| 43a | CH₃ | —(CH₂)₃C₆H₄(p-NMe₂.HCl) | H | 36.0 |
| 98 | CH₃ | —(CH₂)₃C₆H₄(p-CF₃) | H | 1.8 |
| 99 | CH₃ | —(CH₂)₃C₆H₄(p-OMe) | H | 17.4 |
| 100 | CH₃ | —(CH₂)₃C₆H₄(3,5-diF) | H | 11.0 |
| 101 | CH₃ | —(CH₂)₃C₆H₄(3,5-diCF₃) | H | 0.3 |
| 102 | ⁿPr | H | H | 70.8 |
| 103 | ⁿBu | H | | 151.8 |
| 104 | (CH₂)₂Ph | H | H | 37.7 |
| | Amphotericin B | — | — | 0.01 |
| | Pentamidine | — | — | 1.35 |

^{a}Activity was measured using luciferase assay as described in the text.^{29a} Activity for 83-92 was measured for the racemic mixture.

TABLE 4

| | |
|---|---|
| 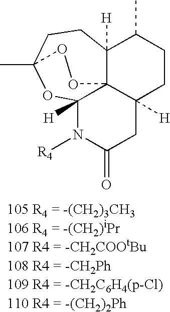 | 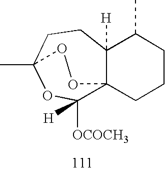 |
| 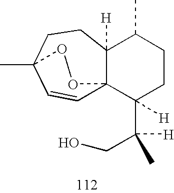 | 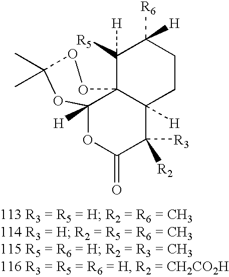 |

| No. | IC₅₀ (μM)^{a} |
|---|---|
| 105 | 44.5 |
| 106 | 185.5 |
| 107 | 27.5 |
| 108 | 56.0 |
| 109 | 25.5 |
| 110 | 32.3 |
| 111 | 148.0 |
| 112 | 131.4 |
| 113 | NA^{b} |
| 114 | 158.3 |
| 115 | 203.5 |
| 116 | NA^{b} |

^{a}Activity was measured using luciferase assay as described in the text.^{29a}
^{b}Not active up to 125 μg/ml.

TABLE 5
Antimalarial activity of new artemisinin analogues.^{a}

| | P. falciparum (D6 Clone) | P. falciparum (W2 Clone) |
|---|---|---|
| No | IC₅₀ (nM) | IC₅₀ (nM) |
| 52 | 14.9 | 14.9 |
| 26 | 4.7 | 1.3 |
| 27 | 10.4 | 3.4 |
| 28 | 43.9 | 50.8 |
| 30 | 30.13 | 20 |
| 33 | 7.9 | 4.7 |
| 34 | 18.8 | 16 |
| 35 | 12.6 | 8.2 |
| 38 | 27.8 | 11.3 |
| 37a | 38.7 | 62.3 |
| 43a | 16.5 | 14.3 |
| Artemisinin | 17.0 | 14.5 |
| Chloroquine | 16.5 | 232.6 |
| Mefloquine | 169.5 | 194.6 |

^{a}Activity was measured using the pLDH assay as described in text.²⁸

An analysis of Table 1-Table 4 clearly shows that substitution at the C-9β position brings about a significant improvement in the activity compared to artemisinin (1). It is more pronounced in the case of phenethyl derivatives with halogens (e.g. 71, 72 and 34), SMe (28), and NMe₂ (38) groups on the aromatic ring. Even though the lactols of this class of compounds were active, the cytotoxicity exhibited by a few of them was discouraging. The 10-deoxo derivatives (93-104, 43a) were expected to be more stable compared to the corresponding acetals, which are relatively unstable due to possible solvolysis or metabolism to a masked aldehyde carbonyl (lactols). The 10-deoxo compounds also exhibited superior activity to the corresponding lactones. The requirement of the unique skeletal framework of artemisinin for its activity is evident from the examples 111-116, where one of the rings has been modified. These compounds did not show any noticeable activity against leishmania.

In general, activity against P. falciparum appears to be more pronounced (nanomolar range)^{31, 32} compared to that against L. donovani (micromolar range). However, in all the above cases, a similar profile of activity was shown by the artemisinin derivatives against both the organisms. The substitution at C-3 appears to be an exception. Although, bulky substitution at C-3 were shown to improve the antimalarial activity, in case of antileishmanial potency this substitution does not seem to make a significant contribution (Table 2-5) as exemplified by 77 and 103.

As many of the artemisinin derivatives studied were notably leishmanicidal, and the presence of an endoperoxide has been shown to be necessary for antimalarial action,³⁰ it was logical to study whether the endoperoxide moiety is a key structural feature required for the observed antileishmanial activity. To test this idea, several 1-deoxo derivatives (117-121, FIG. 5) were prepared by hydrogenolysis of an ethyl acetate solution of the corresponding peroxide compound. Subsequent rigorous HPLC purification was conducted to ensure that all traces of peroxide were removed.

[image]

These 1-deoxo derivatives were tested for their influence on parasitic growth. None of the non-peroxides showed activity in the concentration range tested (up to 125 μg/ml) which clearly emphasizes the role of the peroxide group for the antileishmanial effect. The mechanism of action of artemisinin against P. falciparum is believed to involve free radical intermediates generated in a reaction cascade activated by heme. The leishmania parasite is also known to utilize heme and free iron (II) ³³ for its survival, which points towards a potentially similar mode of action.

It is also interesting to note the effect of C-9α substitution on both antimalarial and antileishmanial activity. In general, compounds with C-9α substitution appear to have lower antimalarial (Table 5), as well as antileishmanial activity (Table 2-5). This is exemplified by compounds 27 and 37a, which show relatively lower potency compared to the corresponding C-9β substituted compounds 26 and 38, respectively. This could be due to masking of the peroxide group by the substituted aromatic ring when C-9 is in the α orientation.

A primary consideration is oral potency and duration of action. Variation in the side-chain is important to achieve fine-tuning of the pharmacokinetic profile. The new artemisinin analogs 47, 52-56, and 59 offers much promise to achieve this goal.

### Experimental Section

All reactions were carried out under an argon atmosphere with dry, freshly distilled solvents under anhydrous conditions, unless otherwise stated. Thin layer chromatography (tlc) was performed on precoated silica gel G and GP Uniplates from Analtech. The plates were visualized with a 254-nm UV light. Flash chromatography was carried out on silica gel 60 [Scientific Adsorbents Incorporated (SAI), particle size 32-63 μm, pore size 60 Å]. Melting points were noted on a FP62 mettler Toledo apparatus and are uncorrected. ¹H NMR and ¹³C NMR spectra were recorded on a Bruker DPX 300 operating at 400 MHz and 100 MHz, respectively. The chemical shifts are reported in parts per million (ppm) downfield from tetramethylsilane, and J-values are in Hz. IR spectra were recorded using a Thermo Nicolet IR 300 Fr/IR spectrometer on a germanium crystal plate as neat solids or liquids. Low-resolution mass spectra were recorded using a Waters Micromass ZQ LC-Mass system or Thermo-Finnigin AqA by direct injection in ESI+mode. GC/MS data were obtained with a Hawlett Packard 5890A gas chromatographic instrument. The high-resolution mass spectra (BS) were recorded on a Micromass Q-Tof Micro with lock spray source. Optical rotations were measured on an Autopol® IV automatic polarimeter from Rudolph Research Analytical. Elemental analysis data were obtained with a Perkin Elmer series II 2400 CHNS/O analyzer.
Preparation of (18) from artemisinin (1)

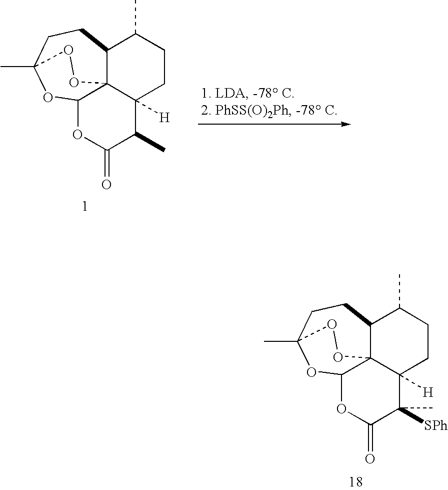

In a flame-dried, 10 mL round-bottom flask equipped with an argon line, 1 (282 mg, 1 mmol, 1 eq.) was placed and was dissolved in THF (2 mL). This solution was added via cannula to a solution of LDA (1.2 eq, 1.2 mmol) in dry THF (2 mL) at −78° C. After 30 minutes, a solution of S-phenylbenzenethiosulfonate (325 mg, 1.3 mmol, 1.3 eq) in THF was added. The reaction mixture was left stirring for another 30 minutes at temperature of −78° C. The reaction was quenched with distilled water (2 mL) and then allowed to warm to room temperature. The reaction mixture was diluted with H₂O (5 mL) and extracted with EtOAc (3×25 mL). The combined organic layers were then washed with brine (1×25 mL), dried over MgSO₄, filtered and concentrated in vacuo. The crude oil was purified via flash column chromatography eluting with 20% ethyl acetale/hexanes. Compound 18 (255 mg) was isolated in 67% yield.

(+)-Octahydro3,6α-dimethyl-3,12-epoxy-9β-(phenylthio)-12H-pyrano[4,3j]-1,2-benzodioxepin-10(3H)-one (18) (67% yield). m.p. 171-174° C. ¹H NMR (CDCl₃): δ7.47 (d, 2H J=8 Hz), 7.38 (d, 1H J=1.2 Hz) 7.32 (t, 2H, J=7.28 Hz ) 6.02 (s, 1H), 2.42 (d, 1H J=2.92 Hz), 2.34 (m, 1H), 2.06 (d, 1H J=3.6 Hz), 2.03 (d, 1H J=3.8 Hz), 1.92 (d, 1H J=3.9 Hz), 1.81-1.74 (m, 2H), 1.53 (s, 3H), 1.43(s, 3H), 1.00 (m, 1H) 0.99 (d, 3H, J=5.0 Hz). ¹³CNMR (CDCl₃): δ171.74, 138.55, 130.35, 128.99, 105.44, 94.60, 82.71, 54.28, 51.51, 50.30, 37.67, 36.38, 34.75, 29.67, 26.19, 26.02, 25,05, 20.31 FABMS: m/z 391.2 (M+H). Anal. Calcd. for: C₂₁H26O₅S C, 64.59, H, 6.79 Found: C, 64.27, H, 6.51.
Preparation of artemisitene (17) from 18.

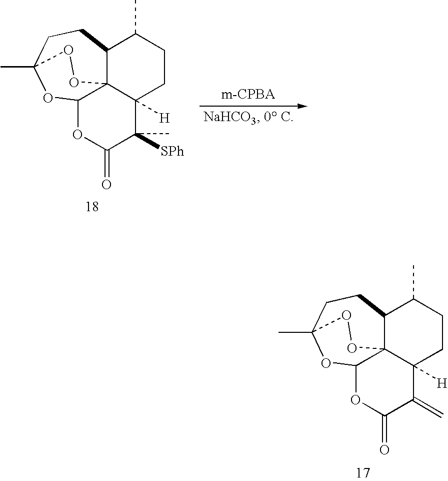

In a flame-dried 10 mL round-bottom flask equipped with an argon line, 35 (30 mg, 0.1 mmol, 1 eq.) was placed and was dissolved in CH₂Cl₂ (2 mL). This solution was added via cannula to a solution of m-CPBA (1.05 eq, 0.11 mmol) in dry CH₂Cl₂ (2 mL) at −78° C. After 6 hours at −78° C., the reaction was allowed to warm to room temperature. The reaction mixture was diluted with H₂O (5 mL), washed with a saturated solution of NaHCO₃ and extracted with EtOAc (3×25 mL). The combined organic layers were then washed with brine (1×25 mL), dried over MgSO₄, filtered and concentrated in vacuo. The crude oil was purified via flash column chromatography eluting with 20% ethyl acetate/hexanes. Artemisitene (28) (16.8 mg) was isolated in 78% yield.
General Procedure for Preparation of the Alcohols 21a-26a From 21-26.

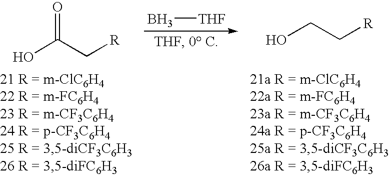

In a flame-dried three-neck 1000 mL round-bottom flask equipped with a reflux condenser and an argon line, the appropriate carboxylic acid (21-26) (1 eq.) was placed and was dissolved in THF. To this solution, a solution of 1M BH₃.THF was added via cannula (2 eq.) at 0° C. The reaction mixture was kept at 0° C. for 4 hours and monitored by TLC. The reaction mixture was diluted with cold H₂O, washed with a saturated solution of NaHCO₃ and extracted with ether. The combined organic layers were then washed with brine (1×25 mL), dried over MgSO₄, filtered and concentrated in vacuo. The residue was purified via flash chromatography on silica gel eluting with 20% ethyl acetate/hexanes to give 21a-26a in 58-88% yield.

m-Chlorophenethyl Alcohol (21a)

¹H NMR (CDCl₃): δ7.21-7.19 (q, 3H), 7.12 (t,1H), 3.76 (t, 2H), 2.78 (t, 2H). 3 (CDCl₃): δ140.90, 134.14, 129.69, 129.06, 127.02, 126.45, 62.96, 38.69. IR (liquid film) 3464, 2948, 2879, 1598, 1574, 1429, 1096, 1047, 958 FT-ICR MS: m/z 157 (M.+H).

m-Fluorophenethyl Alcohol (22a)

¹H NMR (CDCl₃): δ7.19-6.89 (q, 3H), 6.83 (t,1H), 3.76 (t, 2H), 2.74 (t, 2H). ¹³CNMR (CDCl₃): δ162.52, 141.90, 130.14, 127.59, 114.70, 112.70, 63.46, 38.29. IR (liquid film) 3343, 2942, 2878, 1510, 1436, 1416, 1098, 1016, 958 FT-ICR MS: m/z 141 (M.+H).

m-(Trifluoromethyl)phenethyl Alcohol (23a)

¹H NMR (CDCl₃): δ7.30-7.28 (q, 3H) 7.12 (t,1H), 3.86 (t, 2H), 2.74 (t, 2H),. ¹³CNMR (CDCl₃): δ139.95, 132.58, 130.59, 129.00, 127.11, 125.75, 123.30, 63.11, 38.90. IR (liquid film) 3323, 2948, 2879, 1598, 1574, 1429, 1080, 1047, 1000, 968 FT-ICR MS: m/z 191.02 (M.+H).

p-(Trifluoromethyl)phenethyl Alcohol (24a)

¹H NMR (CDCl₃): δ7.26 (d, 2H J=8.6 Hz) 7.04 (d, 2H J=7.8 Hz), 3.91 (t, 2H, J=7.9 Hz), 2.76 (t, 2H, J=8.0 Hz). ¹³CNMR (CDCl₃): δ139.95, 132.58, 130.59, 129.00, 127.11, 125.75, 123.30, 63.11, 38.90. IR (liquid film) 3463, 2948, 2879, 1598, 1574, 1429, 1080, 1047, 1000, 968 FT-ICR MS: m/z 191.02 (M. +H).

3,5-Difluorophenethyl Alcohol (25a) ¹H NMR (CDCl₃): δ6.63 (s, 2H), 6,03 (s, 1H), 3.88 (t, 2H, J=8.0 Hz), 2.78 (t, 2H J=8.8 Hz). ¹³CNMR (CDCl₃): δ163.52, 142.90, 110.70, 99.70, 65.46, 38.71. IR (liquid film) 3453, 2942, 2878, 1510, 1436, 1416, 1098, 1010, 958 FT-ICR MS: m/z 159.23 (M. +H).

3,5-Di(trifluoromethyl)phenethyl Alcohol (26a)

¹H NMR (CDCl₃): δ7.38 (s, 11), 7,23 (s, 2H), 3.82 (t, 2H, J=8.2 Hz), 2.75 (t, 2H J=8.7 Hz). ¹³CNMR (CDCl₃): δ140.90, 131.3, 128.05, 119.70, 65.76, 39.31. IR (liquid film) 3468, 2947, 2878, 1530, 1436, 1416, 1098, 1010, 958 FT-ICR MS: m/z 257.18 (M. +H).
General Procedure for the Preparation of the Iodides 21b-26b from 21a-26a.

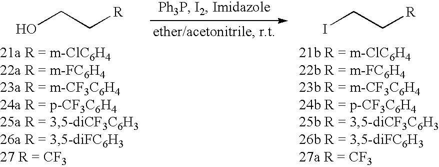

In a flame-dried 250 mL round-bottom flask equipped with an argon line, the appropriate alcohol (21a-26a) (1 eq.) was placed and was dissolved in ether/acetonitrile (3:1). To this solution, triphenylphosphine (3 eq.), imidazole (3 eq.) and iodine (3 eq.) were added in this order. The reaction mixture was kept at room temperature for 1 hours and monitored by TLC. The reaction mixture was filtered and washed with ether. The organic layers were then washed with brine, dried over MgSO₄, filtered and concentrated in vacuo. The residue was purified via flash chromatography on silica gel eluting with 1% ethyl acetate/hexanes to give 21b-26b in 58-88% yield.

-Chlorophenethyl iodine (21b)

¹H NMR (CDCl₃): δ7.21-7.19 (q, 3H), 7.12 (t, 1H), 3.52 (t, 2H), 3.28 (t, 2H). ¹³CNMR (CDCl₃): δ140.90, 134.14, 129.69, 129.06, 127.02, 126.45, 38.69, 11,27. IR (liquid film) 2948, 2879, 1598, 1574, 1429 FT-ICR MS: m/z 267.52 (M. +H).

m-Fluorophenethyl iodine (22b) ¹H NMR (CDCl₃): δ7.19-6.89 (q, 3H), 6.79 (t, 1H), 3.47 (t, 2H), 3.16(t, 2H). ¹³CNMR (CDCl₃): δ162.02, 141.90, 130.14, 127.39, 114.70, 112.70, 38.05, 11.29. IR (liquid film) 2942, 2878, 1510, 1436, 1416, 1098 FT-ICR MS: m/z 251 (M. +H).

m-(Trifluoromethyl)phenethyl iodine (23b)

¹H NMR (CDCl₃): δ7.30-7.28 (q, 3H) 7.12 (t, 1H), 3.56 (t, 2H), 3.24 (t, 2H). ¹³CNMR (CDCl₃): δ139.95, 132,58, 130.59, 129.00, 127.11, 125.75, 123.32, 38.90, 10.89. IR (liquid film) 2948, 2879, 1598, 1574, 1429, 1080, 1047 FT-ICR MS: m/z 301 (M.+H).

p-(Trifluoromethyl)phenethyl iodine (24b) ¹H NMR (CDCl₃): δ7.46 (d, 2H J=8.6 Hz) 7.14 (d, 2H J =7.8 Hz), 3.41 (t, 2H, J=7.7 Hz), 3.22 (t, 2H, J=8.0 Hz). ¹³CNMR (CDCl₃): δ139.95, 132.58, 130.59, 129.00, 127.11, 125.75, 123.30, 38.93, 11.02. IR (liquid film) 2948, 2879, 1598, 1574, 1429, 1080, 1047 FT-ICR MS: m/z 301.02 (M. +H).

3,5-Difluorophenethyl iodine (25b)

¹H NMR (CDCl₃): δ6.60 (s, 2H), 6,30 (s, 1H), 3.48 (t, 2H, J=7.8 Hz), 3.16 (t, 2H J=7.9 Hz). ¹³CNNR (CDCl₃): δ166.62, 143.40, 110.50, 99.70, 39.71, 12.5. IR (liquid film) 2942, 2878, 1510, 1436, 1416, 1098 FT-ICR MS: m/z 269.06 (M. +H).

3,5-Di(trifluoromethyl)phenethyl iodine (26b)

¹H NMR (CDCl₃): δ7.46 (s, 1H), 7,31 (s, 2H), 3.46 (t, 2H, J=7.2 Hz), 3.12 (t, 2H J=7.6 Hz). ¹³CNMR (CDCl₃): 140.97, 131.37, 128.65, 119.72, 39.31, 11.5. IR (liquid film) 2947, 2878, 1530, 1436, 1416, 1098, 1010, 958 FT-ICR MS: m/z 368.08 (M. +H).

General Procedure for the Radical Induced Michael Addition Approach: Preparation of 9β-Artemisinin Analogs 29-34 from Artemisitene (17)

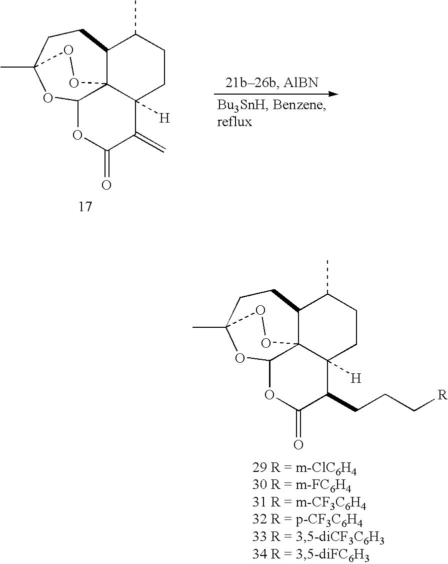

To a solution of the artemisitene (28) (1 eq.) in dry benzene were added the iodo derivative 21b-26b (1.5 eq.), and AIBN at 25° C. The reaction mixture was heated under reflux temperature. Tributyltin hydride was added over a period of 8 hours. After the completion of addition, the reaction mixture was cooled to room temperature. The solvent was evaporated to dryness, and diethyl ether followed by a saturated solution of potassium fluoride was added. The solution-was left stirring at 25° C. for 12 hours. The solution was filtered, washed with water, and evaporated to dryness. The crude product was purified by flash chromatography on silica gel using a mixture of 15% ethyl acetate/hexanes as eluent to give a mixture of β isomer 29-34 in 35-40% yield and α isomer in 60-65% yield.
Conjugate addition of 1,1,1-trifluoropropanylmagnesium bromide to artemisitene (17)

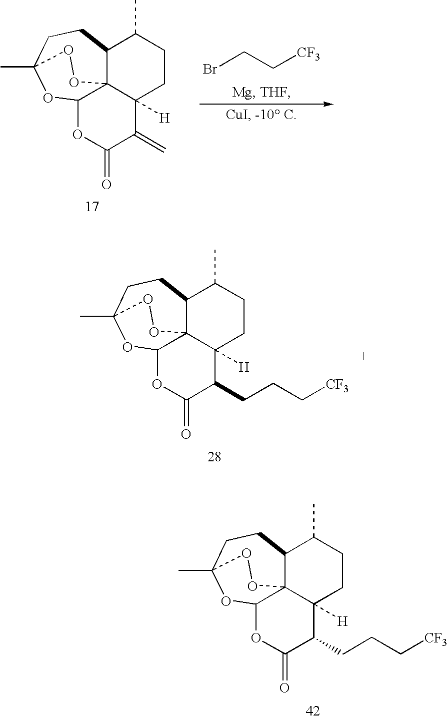

A solution of artemisitene (1 eq.) and CuI (catalytic) in THF at −10° C. under argon was treated dropwise with the appropriate Grignard reagents (1.10 eq.). The mixture was at −10° C. for a further 1 hour after which saturated aqueous NH₄Cl solution was added and the mixture was allowed to warm up to room temperature. After separation of the organic layer, the aqueous solution was extracted with Et2O. The combined organic layer was washed with brine, dried with MgSO₄ and concentrated in vacua. The residue was then purified by flash chromatography on silica gel using a mixture of 15% ethyl acetate/hexanes as eluent to afford a mixture of β isomer 28 in 38% yield and a isomer 42 in 58% yield.
General Procedure for the Conversion of α-epimers to the 9β-artemisinin Analogs 28-34 Using DBU: Preparation of the 10-dihydro 9β-artemisinin Analogs

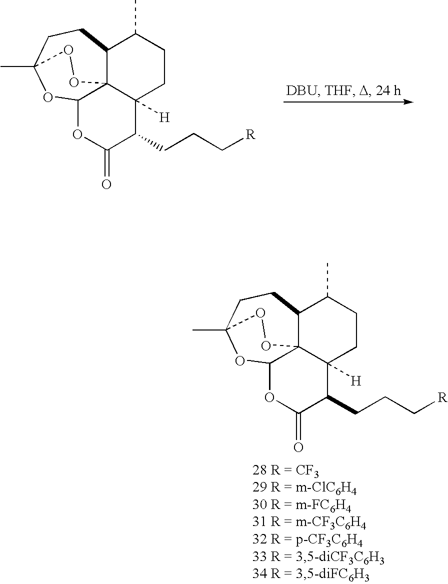

To a solution of 9α-artemisinin analogs (1 eq.) in dry THF was added DBU (2 eq.) at 25° C. The reaction mixture was refluxed for 24 hours. Afler cooling to room temperature, water was added, and the organic material was extracted with ethyl acetate. The solution was dried over MgSO₄, filtered, and evaporated to dryness. The crude product was purified by silica gel column chromatography using 15% ethyl acetate/hexanes as eluent to give the β isomer 28-34 in 50-70% yield.

(+)-Octahydro-3,6α-dimethyl-3,12-epoxy-9β-(3′-(trifluoromethyl)propyl)-12H-pyrano[4,3j]-1,2-benzodioxepin-10(3H)-one (28) (58% yield)

m.p. 93-94° C. ¹H NMR (CDCl₃): δ5.91 (s, 1H), 2.23 (dd, 2H J=1.5 Hz) 2.11-2.03 (m, 6H), 1,74-1.66(m, 6H), 1.47 (s, 3H), 1.01 (d, 3H, J=5.0 Hz). ¹³CNMR (CDCl₃). δ171.73, 105.76, 94.21, 80.58, 50.87, 45.22, 43.59, 37.93, 36.30, 34.43, 33.74, 31.96, 25.84, 25.09, 20.52, 20.26 IR (KBr): 2930, 1740 cm⁻¹. FABMS: m/z 379.45 (M +H). Anal Calcd. for: C₁₈H₂₅O₅F₃ C, 57.14, H, 6.66 Found: C, 57.17, H, 6.65.

(+)-Octahydro-3,6α-dimethyl-3,12-epoxy-9β-(3′-(m-chlorophenyl)propyl)-12H-pyrano[4,3j]-1,2-benzodioxepin-10(3H)-one (29) (62% yield)

m.p.116-117° C. ¹H NMR (CDCl₃): δ7.23 (d, 2H, J=8.2 Hz), 7.09 (d, 2H, J=8.3 Hz), 5.83 (s, 1H), 3.14-3.24 (m, 1H), 2.52-2.72 (m, 2H), 2.34-2.48 (m, 1H), 1.96-2.09 (m, 3H), 1.67-1.82 (m, 4H), 1.44 (s, 3H), 0.98 (d, 3H, J=5.7 Hz). ¹³CNMR (CDCl₃): δ171.76, 146.47, 129.13, 127.09, 125.71, 122.45, 106.42, 93.92, 79.60, 50.81, 43.50, 38.09, 37.91, 36.30, 35.89, 33.75, 29.17, 26.87, 25.57, 25.44, 25.04, IR (KBr): 2951, 2925, 1740, 1491, 1112, 1031, 1000 cm⁻¹. FABMS: m/z 427(M+Li). Anal Calcd. for C₂₃H₂₉O₅Cl: C, 65.63, H, 6.89. Found: C, 65.79, H, 6.89.

(+)-Octabydro-3,6α-dimethyl-3,12-epoxy-9β-(3′-(m-fluorophenyl)propyl)-12H-pyrano[4,3j]-1,2-benzodioxepin-10(3H)-one (30) (52% yield)

m.p. 152-153 ° C. ¹H NMR (CDCl₃): δ7.15-6.93 (m, 4H), 5,83 (s, 1H), 3.22 (ddd, 1H, J=5.5, 2.7, 7.0 Hz), 2.65 (ddd, 1H, J=6.2, 8.0, 10.4 Hz), 2.58 (ddd, 1H, J=6.2, 7.6, 8.4 Hz), 2.42 (ddd, 1H, J=4.4, 13.0, 15.1 Hz), 1.60-2.19 (m, 9H), 1.44 (s, 3H), 1.25-1.50 (m, 4H), 1.05 (m, 1H), 0.98 (d, 3H, J=5.8 Hz). ¹³ CNMR(CDCl₃): δ172.84, 143.47, 133.08, 128.6, 127.09, 125.71, 122.45, 106.42, 93.92, 79.60, 50.81, 43.50, 38.09, 37.91, 36.30, 35.89, 33.75, 29.17, 26.87, 25.57, 25.44, 23.84, 19.92. IR (KBr): 1736, 1382, 1261, 1184, 1116, 881, 850, 831, 798 cm⁻¹. FABMS, m/z: 411 (M +Li). Anal Calcd. for C₂₃H₂₉O₅F: C, 68.28, H, 7.23. Found: C, 68.42, H, 7.42.

(+)-Octahydro-3,6α-dimethyl-3,12-epoxy-9β-(3′-(m-trifluoromethylphenyl)propyl)-12H-pyrano[4,3j]-1,2-benzodioxepin-10(3H)-one (31)(62% yield)

m.p. 112-114 ° C. ¹H NMR (CDCl₃): δ7.51 (d, 2H) 7.27 (d, 2H), 5,83 (s, 1H), 3.21 (ddd, 1H, J=5.3, 2.5, 7.0 Hz), 2.65 (ddd, 1H, J=6.1, 8.3, 10.2 Hz), 2.61 (ddd, 1H, J=6.2, 7.6, 8.4 Hz), 2.42 (ddd, 1H, J=4.4, 13.0, 15.1 Hz), 1.60-2.21 (m, 9H), 1.44 (s, 3H), 1.25-1.40 (m, 4H), 1.05 (m, 1H), 0.96 (d, 3H, J=5.6 Hz). ¹³ CNMR (CDCl₃): δ171.79, 147.47, 131.56, 128.6, 127.09, 125.71, 122.45, 119.6, 104.42, 93.92, 79.60, 50.81, 43.50, 38.09, 37.91, 36.30, 35.93, 33.75, 29.17, 26.77, 25.57, 25.28, 23.81, 20.20. IR (KBr): 1734, 1383, 1261, 1187, 1116, 881, 856, 831, 796 cm⁻¹. FABMS, m/z: 461 (M +Li). Anal Calcd. for C₂₄H₂₉O₅F₃: C, 63.43, H, 6.43. Found: C, 63.37, H, 6.56.

(+)ctahydro-3,6α-dimethyl-3,12-epoxy-9β-(3′-(p-trifuoromethylphenyl)propyl)-12H-pyrano[4,3j]-1,2-benzodioxepin-10(3H)-one (32) (67% yield)

m.p. 153-154° C. ¹H NMR (CDCl₃): δ7.52 (d, 2H), 7.29 (d, 2H), 5,83 (s, 1H), 3.22 (ddd, 1H, J=5.5, 2.7, 7.0 Hz), 2.65 (ddd, 1H, J=6.2, 8.0, 10.4 Hz), 2.58 (ddd, 1H, J=6.2, 7.6, 8.4 Hz), 2.42 (ddd, 1H, J=4.4, 13.0, 15.1 Hz), 1.60-2.19 (m, 9H), 1.44 (s, 3H), 1.25-1.50 (m, 4H), 1.05 (m, 1H), 0.98 (d, 3H, J=5.8 Hz). ¹³ CNMR (CDCl₃): δ171.76, 146.47, 129.09, 125.69, 125.65, 105.81, 93.88, 79.59, 50.48, 43.50, 38.09, 37.91, 36.30, 35.93, 33.95, 29.07, 26.76, 25.55, 25.24, 23.71, 20.18. IR (KBr): 1736, 1382, 1261, 1184, 1120, 886, 850, 831, 798 cm⁻¹. FABMS, m/z: 461 (M+Li). Anal Calcd. for C₂₄H₂₉O₅F₃: C, 63.43, H, 6.43. Found: C, 63.35, H, 6.59.

(+)-Octahydro-3,6α-dimethyl-3,12-epoxy-9β-(3′-(3,5-bistrifluoromethylphenyl)propyl)-12H-pyrano[4,3j]-1,2-benzodioxepin-10(3H)-one (33) (58% yield)

m.p. 109-110° C. ¹H NMR (CDCl₃): δ7.46 (d, 1H), 7.31 (d, 2H) 5,83 (s, 1H), 3.22 (ddd, 1H, J=5.5, 2.7, 7.0 Hz), 2.65 (ddd, 1H, J=6.2, 8.0, 10.4 Hz), 2.58 (ddd, 1H, J=6.2, 7.6, 8.4 Hz), 2.42 (ddd, 1H, J=4.4, 13.0, 15.1 Hz), 1.60-2.19 (m, 9H), 1.44 (s, 3H), 1.25-1.50 (m, 4H), 1.05 (m, 1H), 0.98 (d, 3H, J=5.8 Hz). ¹³ CNMR (CDCl₃): δ176.00, 140.07, 131.00, 112.45, 105.14, 92.46, 78.89, 50.93, 44.89, 39.62, 39.45, 35.86, 31.17, 29.24, 28.18, 27.97, 26.53, 23.97, 21.12. IR (KBr): 1740, 1382, 1261, 1184, 1116, 881, 850, 831, 798 cm⁻¹. FT-ICR MS, m/z: 529.5 (M+H). Anal Calcd. for C₂₃H₂₈O₅F₂: C, 57.47; H, 5.40. Found: C, 57.45, H, 5.42.

(+)-Octahydro3,6α-dimethyl-3,12-epoxy-9β-(3′-(3,5-difluorophenyl)propyl)-12H-pyrano[4,3j)-1,2-benzodioxepin-10(3H)-one (34) (52% yield)

m.p. 100-104° C. ¹H NMR (CDCl₃): δ7.15-6.93 (m, 3H), 5,83 (s, 1H), 3.22 (ddd, 1H, J=5.5, 2.7, 7.0 Hz), 2.65 (ddd, 1H, J=6.2, 8.0, 10.4 Hz), 2.58 (ddd, 1H, J=6.2, 7.6, 8.4 Hz), 2.42 (ddd, 1H, J=4.4, 13.0, 15.1 Hz), 1.60-2.19 (m, 9H), 1.44 (s, 3H), 1.25-1.50 (m, 4H), 1.05 (m, 1H), 0.98 (d, 3H, J=5.8 Hz). ¹³ CNMR (CDCl₃): δ173.90, 150.07, 142.00, 112.45, 105.14, 100.02, 90.46, 79.60, 50.93, 44.60, 39.62, 39.47, 35.86, 31.17, 29.24, 28.18, 27.91, 26.53, 23.97, 20.13. IR (KBr): 1736, 1382, 1261, 1184, 1116, 881, 850, 831, 798 cm⁻¹. FT-ICR MS, m/z: 429 (M+H). Anal Calcd. for C₂₃H₂₈O₅F₂: C, 65.39; H, 6.68. Found: C, 65.42, H, 6.66.

General Procedure for Reduction of the Lactone 28-34 Derivative to Lactol 28a-34a Using DIBAL: Preparation of the 10-dihydro 9β-artemisinin Analogs.

To a stirred solution of 28-34 (1.0 equiv.) in dry CH₂Cl₂ at −78° C. was added 1M DIBAL in CH₂Cl₂ (1.1 eq.) After 1 hour the reaction was quenched with saturated NaHCO₃, diluted with CH₂Cl₂, and allowed to warm to room temperature.

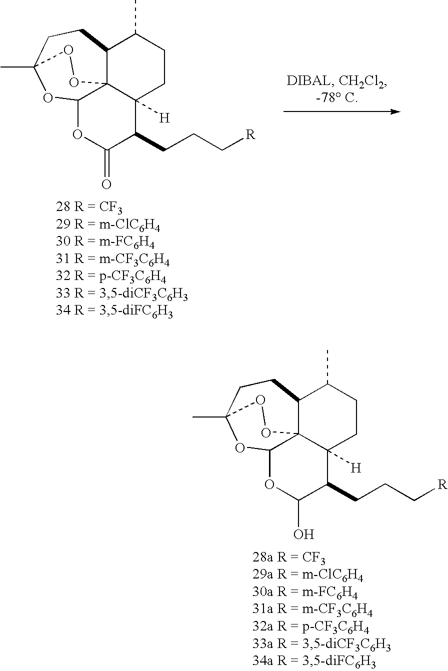

The mixture was diluted with CH₂Cl₂ and washed with 10% HCl/saturated NH₄Cl (1:15 v/v). The CH₂Cl₂ layer was then dried over MgSO₄, filtered, and concentrated in vacuo to give 46a-52a as a white solid in 89-96% yield.

General procedure for reduction of the lactol 28a-34a derivative to the pyran 35-41 using Et₃SiH/BF₃OEt₂: preparation of 10-deoxo 9β-artemisinin analogs.

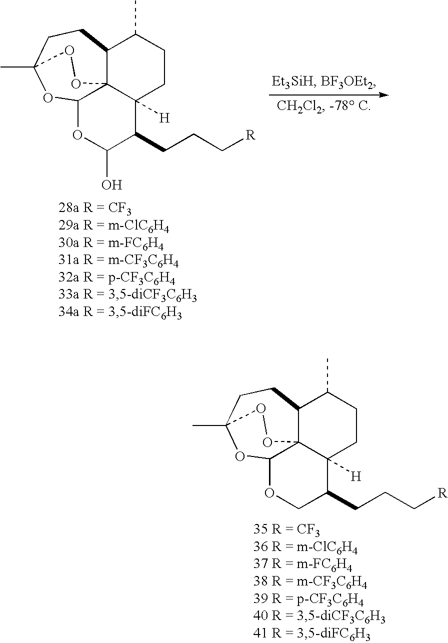

To a stirred solution of 28a-34a (1 eq.) in dry CH₂Cl₂ at −78° C., Et₃SiH (4 eq.) was added. The reaction was stirred for 10 min. and BF₃OEt₂ (1.5 eq.) was added. The resultant solution was allowed to stir 3 hours at −78° C. After 5 hours, the reaction was quenched at −78° C. with pyridine (8 eq.) and was allowed to warm to room temperatures. The reaction mixture was poured into aqueous saturated NH₄Cl, and extracted wvith EtOAc. The combined organic layers were washed with NH₄Cl, dried (MgSO₄), and concentrated in vacuo to give a white solid. The crude products were purified by flash chromatography on silica gel (80:20 EtOAc/hexanes) to give 35-41 as a pure white crystalline compounds, in 55-85% yield.

(+)-Octahydro-3,6α-dimethyl-3,12-epoxy-9β-(3′-(trifluoromethyl)propyl)-12H-pyrano[4,3j]-1,2-benzodioxepin (35)(85% yield)

m.p. 125-128° C. ¹NMR (CDCl₃): δ5.53 (s, 1H), 4.02 (s, 1H), 3.82 (d, 2H, J=18 Hz), 2.31-2.25 (m, 2H), 2.12-2.03 (m, SH), 1.93-189 (m, 2H), 1.71-1.69 (m, 2H), 1.62-1.49 (m, 8H), 1.41 (s, 3H), 1.29-125 (m, 2H), 0.95 (d, 3H, J=5.0 Hz). FT-ICR MS: m/z 365 (M+H). Anal Calcd. for C₁₈H₂₇O₄F₃: C, 59.33, H, 7.47. Found: C, 59.22, H, 7.43.

(+)-Octahydro-3,6α-dimethyl-3,12-epoxy-9β-(3′-(m-chlorophenyl)propyl)-12H-pyrano[4,3j]-1,2-benzodioxepin (36)(85% yield)

m.p. 92-93° C. ¹H NMR (CDCl₃): δ7.00-7.7.15 (m, 4H), 5.19 (s, 1H), 3.56 (ddd, 1H, J=1.3, 4.3, 7.2 Hz), 3.44 (dd, 1H, J=11.7, 11.7 Hz), 2.60 (ddd, 2H, J=5.1, 7.4, 7.4 Hz), 2.43-2.53 (m, 1H), 2.37 (ddd, 1H, J=4.1, 13.4, 14.7 Hz), 2.01 (ddd, 1H, J=3.0, 4.8, 14.7 Hz), 1.83-1.93 (m, 1H), 1.62 (dd, 2H, J 7.8, 7.8 Hz), 1.42 (s, 3H), 1.25 (dd, 2H, J=6.5, 11.1 Hz), 0.95 (d, 3H, J=6.2 Hz). IR (KBr): 2920, 2915, 2860, 1490, 1452, 1090, 1067 cm⁻¹. FABMS: m/z 413 (M+Li). Anal Calcd. for C₂₃H₃₁O₄C1: C, 67.88, H, 7.68. Found: C, 67.45, H, 7.55.

(+)-Octahydro-3,6α-dimethyl-3,12-epoxy-9β-(3′-(m-fluorophenyl)propyl)-12H-pyrano[4,3j]-1,2-benzodioxepin (37) (82% yield)

m.p. 76-77° C. ¹H NMR (CDCl₃): δ7.12-7-6.92 (m, 4H), 5.19 (s, 1H), 3.78 (dd, 1H, J=3.2, 10.9 Hz), 3.43 (dd, 1H, J=11.7, 11.7 Hz), 2.61-2.32 (m, 4H), 2.15-1.81 (m, 2H), 1.71-1.45 (m, 7H), 1.43 (s, 3H), 1.41-1.04 (m, 5H), 0.97 (d, 3H, J=6.3 Hz). IR (KBr): 1633, 1511, 1454, 1216, 1193, 1159, 1126, 910, 877, 829, 761, 736 cm⁻¹. FABMS m/z: 397 (M+Li). Anal Calcd. for C₂₃H₃₁O₄F: C, 70.73, H, 8.01. Found: C, 70.83, H, 8.21.

(+)-Octahydro-3,6α-dimethyl-3,12-epoxy-9β-(3′-(m-trifluoromethylphenyl)propyl)-12H-pyrano[4,3j]-1,2-benzodioxepin (38) (80% yield)

m.p. 93-94° C. ¹H NMR (CDCl₃): δ7.12-7-6.92 (m, 4H), 5.19 (s, 1H), 3.76 (dd, 1H, J=3.2, 10.9 Hz), 3.47 (dd, 1H, J=11.6, 11.5 Hz), 2.61-2.36 (m, 4H), 2.13-1.82 (m, 2H), 1.69-1.48 (m, 7H), 1.43 (s, 3H), 1.41-1.04 (m, 5H), 0.97 (d, 3H, J=6.3 Hz). IR (KJr): 1636, 1516, 1457, 1212, 1197, 1158, 1126, 910, 877, 829, 767, 739 cm⁻¹. FABMS m/z: 447 (M+Li). Anal Calcd. for C₂₄H₃₁O₄F₃: C, 65.44, H,7.09. Found: C, 65.13, H, 6.91.

(+)-Octahydro-3,6α-dimethyl-3,12-epoxy-9β-(3′-(p-trifluoromethylphenyl)propyl)-12H-pyrano[4,3j]-1,2-benzodioxepin (39) (80% yield)

m.p. 79-80° C. ¹H NMR (CDCl₃): 7.51 (d, 2H), 7.28 (d, 2H), 5.19 (s, 11H), 3.76 (dd, 1H, J=3.2, 10.9 Hz), 3.47 (dd, 1H, J=11.6, 11.5 Hz), 2.61-2.36 (m, 4H), 2.13-1.82 (m, 2H), 1.69-1.48 (m, 7H), 1.43 (s, 3H), 1.41-1.04 (m, 5H), 0.97 (d, 3H, J=6.3 Hz). IR (KBr): 1636, 1516, 1457, 1212, 1197, 1158, 1126, 910, 877, 829, 767, 739 cm⁻¹. FABMS m/z: 447 (M+Li). Anal Calcd. for C₂₄H₃₁O₄F₃: C, 65.44, H,7.09. Found: C, 65.91, H, 7.37.

(+)-Octahydro-3,6α-dimethyl-3,12-epoxy-9β-(3′-(3,5-bistrifluoromethylphenyl)propyl)-12H-pyrano[4,3j]-1,2-benzodioxepin (40) (76% yield)

m.p. 109-110° C. ¹H NMR (CDCl₃): δ7.46 (d, 1H), 7.31 (d, 2H) 5,83 (s, 1H), 3.72 (dd, 1H, J=3.7, 9.0 Hz), 2.65 (dd, 1H, J=6.2, 8.0, 10.4 Hz), 2.58 (ddd, 1H, J=6.2, 7.6, 8.4 Hz), 2.42 (ddd, 1H, J=4.4, 13.0, 15.1 Hz), 1.60-2.19 (m, 9H), 1.44 (s, 3H), 1.25-1.50 (m, 4H), 1.15 (m, 1H), 0.96 (d, 3H, J=5.8 Hz). FT-ICR MS, m/z: 509.50 (M+H). Anal Calcd. for C₂₅H₃₀O₄F₆: C, 59.05; H, 5.95. Found: C, 59.09, H, 5.97.

(+)-Octahydro3,6α-dimethyl-3,12-epoxy-9β-(3′-(3,5-difluorophenyl)propyl)-12H-pyrano[4,3j]-1,2-benzodioxepin (41) (70% yield).

m.p. 119-121° C. ¹H NMR (CDCl₃): δ7.13-7.23 (m, 3H), 5,81 (s, 1H), 3.75 (dd, 1H, J=3.9, 8.9 Hz), 2.63 (dd, 1H, J=6.3, 7.7, 11.4 Hz), 2.60 (ddd, I1H, J=6.2, 7.6, 8.4 Hz), 2.40 (ddd, 1H, J=4.4, 13.0, 15.1 Hz), 1.70-2.19 (m, 9H), 1.44 (s, 3H), 1.25-1.50 (m, 4H), 1.15 (m, 1H), 0.96 (d, 3H, J=5.8 Hz). FT-ICR MS, m/z: 409.50 (M+H). Anal Calcd. for C₂₃H₃₀O₄F₂: C, 67.63; H, 7.40. Found: C, 67.58, H, 7.36.

General Procedure for the Preparation of Substituted Phenethyl alcohols 40a and 40b.

To a flame-dried, three-neck, 100 mL round-bottomed flask equipped with a condenser and argon line was added the appropriate carboxylic acid (10 mmol) dissolved in dry THF (25 mL). After cooling to 0° C., 1M BH₃.THF (20 mmol) was added drop-wise to the stirred mixture and the reaction was then left at 0° C. for 5h. The reaction mixture was diluted with cold water, washed with a saturated solution of NaHCO₃, and extracted with ethyl acetate (3×25 mL). The combined organic layers were washed with brine (1×25 mL), dried over MgSO₄, filtered, and concentrated under reduced pressure. The residue was purified by flash chromatography on silica gel, eluting with 25% ethyl acetate—hexanes to afford the corresponding alcohols.

p-(N,N-dimethylamino)phenethyl Alcohol (40a). Yield, 90%; mp. 56-57° C.; ¹H NMR (CDCl₃): δ2.77 (t, 2H, J=6.6 Hz), 2.92 (s, 6H, NMe₂), 3.80 (t, 2H, J=6.5 Hz), 6.72 (d, 2H, J=8.53 Hz), 7.10 (d, 2H, J=8.51 Hz). ¹³C NMR (CDCl₃): 5 38.21, 40.93 (NMe₂), 64.01, 113.20 (2C, Ar), 126.43 129.72 (2C, Ar), 149.51; IR (neat): 3284, 2855, 1617, 1527, 1352, 1049, 1021, 804. HRMS (ESI) m/z: Calcd for C₁₀H₁₅NO [M+H]⁺166.1232, found 166.1225 [M+H]⁺.

p-(Methylthio)phenethyl Alcohol (40b). Yield, 94%; mp. 37-38° C.; ¹H NMR (CDCl₃): δ1.82 (s, 1H), 2.48 (s, 3H), 2.83 (t, 2H, J=6.4Hz), 3.83 (t, 2H, J=6.8 Hz), 7.16 (d, 2H, J=8.0 Hz), 7.24 (d, 2H, J=8.0 Hz); ¹³C NMR (CDCl₃): δ16.17, 38.62, 63.57, 127.16 (2C, Ar), 129.57 (2C, Ar), 135.57, 136.20; IR (neat): 3408, 2949, 1495, 1429, 1037, 1021, 808; GCMS (EI) m/z: Calcd. for C₉H₁₂OS (M⁺, 168), found 168 [M⁺].

Triisopropyl silyl ether protection of 4-hydroxy pbenyl acetic acid methyl ester (39c)

To an ice cooled, stirred solution of the phenolic ester 39c (5.0 g, 30 mmol) and imidazole (2.4 g, 35.25 mmol) in dry DMF (20 mL) in a dried round-bottomed flask, triisopropyl silyl chloride (6.6 mL, 30.8 mmol) was added drop-wise, and the solution was stirred for 5 h, slowly warming to room temperature. The mixture was then washed with water (1×25 mL) and extracted with ether (3×30 mL). The combined organic layers were washed with brine (1×25 mL), dried over MgSO₄, and filtered. The solvents were evaporated under reduced pressure. The crude product was purified by flash chromatography over silica gel, eluting with 5% ethyl acetate-hexanes to afford 9.5 g of the product 39c′ as an oily liquid. Yield 98%; ¹H NMR (CDCl₃): δ1.13 (d, 18H, J=7.2 Hz), 1.28 (m, 3H), 3.54 (s, 2H), 3.70 (s, 3H), 6.85 (d, 2H, J=8.4 Hz), 7.15 (d, 2H, J=8.4 Hz); ¹³C NMR (CDCl₃): δ12.68, 17.91, 40.36, 51.90, 119.89, 126.34, 130.17, 155.17, 172.35; IR (neat): 2945, 2868, 1740, 1511, 1266, 1156, 915, 686; GCMS (EI) m/z: Calcd. for C₁₈H₃₀O₃Si (M⁺), found 322 [M⁺].

Preparation of 4(triisopropyl silyloxy) phenyl ethyl alcohol (40c)

To an ice cooled, stirred suspension of lithium aluminum hydride (0.37 g, 9.6 mmol) in dry ether (12 mL) in 50 mL round bottom flask, a solution of 39c′ (1.55 g, 4.8 mmol) in ether (15 mL) was added drop-wise, and the mixture was stirred for 1h, monitoring the reaction by tlc until completion. A saturated solution of sodium sulfate (10 mL) was added to this drop-wise at 0° C. The solids were filtered and washed with ether (3×30 mL). The organic layers were combined, dried over MgSO₄, and filtered. The combined solvents were removed in vacuo. The residue was purified by flash chromatography on a silica gel column using 15% ethyl acetate-hexanes to afford 1.1 g of 40c as an oily liquid. Yield, 83%; ¹H NMR (CDCl₃): δ1.1 (d, 18H, J=7.0 Hz), 1.25 (m, 3H), 2.79 (t, 2H, J=6.6 Hz), 3.81 (t, 2H, J=6.62 Hz), 6.82 (d, 2H, J=8.35 Hz), 7.05 (d, 2H, J=8.34 Hz); ¹³C NMR (CDCl₃): δ12.69, 17.94, 38.40, 63.80, 119.94 (2C, Ar), 129.88 (2C, Ar), 130.71, 154.67; IR (neat): 3330, 2953, 2868, 1605, 1511, 1266, 1046, 914; GCMS (nt/z): Calcd. for C₁₇H₃₀O₂Si (M⁺, 294), Found 294 [M⁺].

Preparation of p-(N,N-dimethylamino) Phenethyl Bromide (41a)

To a dried 10 mL round-bottomed flash under argon at 0° C. was added 40a (0.1 g, 0.6 mmol), CH₂Cl₂ (2 mL) and carbon tetrabromide (0.3 g, 0.9 mmol) followed by drop-wise addition of a solution of triphenylphosphine (0.16 g, 0.6 mmol) in dry CH₂Cl₂ (1 mL). The mixture was warmed to room temperature after 2h, monitoring the reaction by tic for completion. The reaction mixture was then washed with water (1×10 mL), and the aqueous layer was extracted with CH₂Cl₂ (2×5 mL). The combined organic layers were dried over MgSO₄, and filtered. The combined solvents were evaporated under reduced pressure. The residue was purified by flash chromatography on silica gel (˜20 g), eluting with hexanes to afford 0.86 g of 41a. Yield, 63%; mp. 45-46° C.; ¹H NMR (CDCl₃): δ2.93 (s, 6H), 3.07 (t, 2H, J=7.92 Hz), 3.51 (t, 2H, J=7.7 Hz), 6.70 (d, 2H, J=8.56 Hz), 7.09 (d, 2H, J=8.50 Hz); ¹³C NMR (CDCl₃): δ33.83, 38.74, 40.75 (NMe₂), 112.82 (2C, Ar), 126.87, 129.41 (2C, Ar), 149.67; IR (neat); 2900, 1613, 1519, 1352, 1204, 808; HRMS (ESI) m/z: Calcd for C₁₀H₁₄BrN [M+H]⁺228.0388, found 228.0380 [M+H]⁺.

Preparation p-(Methylthio)Phenethyl Bromide (41b) and 4-(triisopropyl silyloxy) Phenethyl Bromide (41c)

In a dried 25 mnL round-bottomed flask under argon was taken a solution of the appropriate alcohol (1 mmol) in ether/acetonitrile (3:1, 10 mL). Triphenyl phosphine (3 equiv) was added to this solution, followed by imidazole (3 equiv) and then bromine (3 equiv), and the mixture was stirred at room temperature for 2h, monitoring the progress by tlc. The reaction mixture was filtered, and washed with ether (2×15 mL). The organic layers were combined, washed with brine (1×25 mL), dried over MgSO₄, and filtered. The combined solvents were evaporated under reduced pressure. The residue was purified by flash chromatography on silica gel, eluting with 1% ethyl acetate-hexanes to afford the corresponding bromides.

41b. Yield, 92%, ¹H NMR (CDCl₃): δ2.48 (s, 3H), 3.12 (t, 2H, J=7.6 Hz), 3.54 (t, 2H, J=7.6 Hz), 7.14 (d, 2H, J=8.11 Hz), 7.22 (d, 2H, J=8.12 Hz); ¹³C NMR (CDCl₃): δ16.42, 33.47, 39.25, 127.35 (2C, Ar), 129.62 (2C, Ar), 136.18, 137.40; IR (neat), 2921, 1495, 1437, 1095, 809; GCMS (EI) m/z:: Calcd. for C₉H₁₁BrS (M⁺, 230), found 230 [M⁺]. 41c Yield, 75%, %; ¹H NMR (CDCl₃): δ1.12 (d, 18H, J=7.6 Hz), 1.27 (m, 3H), 3.09 (t, 2H, J=8.0 Hz), 3.53 (t, 2H, J=7.6 Hz), 6.84 (d, 2H, J=8.40 Hz), 7.06 (d, 2H, J=8.40 Hz); ¹³C NMR (CDCl₃): δ12.69 (3C, CHs), 17.95 (6×CH₃), 33.28, 38.82, 119.99 (2C, Ar), 129.58 (2C, Ar), 131.40, 155.02; IR (neat): 2946, 2864, 1736, 1511, 1266, 914; GCMS EI m/z: Calcd. for C₁₇H₂₉BrOSi (M⁺, 356), found 356 [M⁺].

General Procedure for the radical induced Michael addition of various aryl alkyl or alkyl bromides to Artemisitene

To a solution of artemisitene³² (1 mmol) in dry benzene (30 mL) in a two-necked 100 mL round-bottom flask equipped with a condenser and an argon line, was added the bromide derivative (41a41c, 1.3 mmol) and AIBN (0.1 mmol). The reaction mixture was heated to reflux, and a solution of tributyltin hydride (1.4 mmol) in benzene (20 mL) was added to it via a syringe pump over a period of 8h. After the addition was completed, the reaction mixture was refluxed for another 1 h. The flask was then cooled to room temperature, and the solvent was evaporated under reduced pressure. The residue was diluted with ether (15 mL), and a saturated solution of potassium fluoride (5 mL) was added. The mixture was stirred at room temperature for 12 h, filtered and the filtrate washed with water. The organic layer was dried over anhydrous MgSO₄, filtered, and the combined solvents were evaporated under reduced pressure to give a crude residue, which was purified by flash column chromatography on silica gel using 15% ethyl acetate-hexanes to afford the α- and β-isomers. The α-isomer could be converted to the β-form by refluxing with DBU in THF for 12h.

(+)Octahydro3,6α-dimethyl-3,12-epoxy-9α-(3′-phenylpropyl)-12H-pyrano[4,3j]-1,2-benzodioxepin-10(3H)-one (27).

This analog was prepared by radical-induced Michael addition of phenethyl iodide to artemisitene according to the general procedure described above. Yield, 30%; [α]_{D}²⁶ +92.0 (CHCl₃, c 0.5); ¹H NMR (CDCl₃): δ0.98 (d, 3H, J=5.64 Hz), 1.01 (m, 1H), 1.40 (m, 4H), 1.45 (s, 3H), 1.75 (m, 6H), 2.08 (m, 4H), 2.40 (m, 1H), 2.62 (t, 2H, J=7.3 Hz), 5.90 (s, 1H), 7.17-7.29 (m, 5H); ¹³C NMR (CDCl₃): δ19.91, 24.75, 25.50, 29.70, 31.66, 34.02, 34.12, 35.87, 35.95, 37.57, 42.81, 45.06, 50.54, 80.31, 93.73, 105.29, 125.81, 128.36 (2C, Ar), 128.41 (2C, Ar), 142.22, 171.88; IR (neat): 2929, 2851, 1740, 1458, 1380, 1106, 996; HRMS (ESI) m/z: Calcd. for C₂₃H₃₀O₅Na [M+Na]⁺409.1991, found 409.1978 [M+Na]⁺; Anal. (C₂₃H₃₀O₅. H₂O) C, 68.31, H, 7.29.

(+)-Octabydro-3,6α-dimethyl-3,12-epoxy-9β-(3′-(p-methylthiophenyl)propyl)-12H-pyrano[4,3j]-1,2-benzodioxepin-10(3H)-one (28). Yield, 17%, mp. 164-165° C.; [α]_{D}²⁵ +54.0 (CHCl₃, c 0.5); ¹H NMR (CDCl₃): δ0.99 (d, 3H, J=5.8 Hz), 1.04 (m, 2H), 1.38 (m, 3H), 1.44 (s, 3H), 1.56 (m, 3H), 1.74 (m, 3H), 2.05 (m, 3H), 2.41 (m, 1H), 2.46 (s, 3H), 2.57 (m, 1H), 2.63 (m, 1H), 3.21 (q, 1H), 5.83 (s, 1H), 7.10 (d, 2H, J=8.10 Hz), 7.20 (d, 2H, J=8.11 Hz); ¹³C NMR (CDCl₃): δ16.69, 20.22, 23.66, 25.26, 25.58, 26.62, 29.20, 33.97, 35.52, 36.32, 37.89, 38.07, 43.27, 50.46, 79.60, 93.82, 105.74, 127.52 (2C, Ar), 129.33 (2C, Ar), 135.73, 139.44, 171.80; IR (neat): 2926, 2866, 1739, 1189, 1109, 1000; ESI MS m/z: Calcd. for C₂₄H₃₂O₅S [M+H]⁺433.2048, found 433.3 [M+H]⁺, 865.4 (2M+H]⁺, 887.4 [2M+Na]⁺; Anal. (C₂₄H₃₂O₅S. 0.5 H₂O) C, H.

(+)Octahydro3,6α-dimethyl-3,12-epoxy-9β-(3′-(p-triisopropylsilyloxyphenyl) propyl)-12H-pyranol[4,3j]-1,2-benzodioxepin-10(3H)-one (31). Yield, 22%; [α]_{D}²⁵ +84.0 (CHCl₃, c 0.5); ¹H NMR (CDCl₃): δ0.98 (d, 3H, J=5.27Hz), 1.02 (m, 2H), 1.08 (d, 18H, J=6.98 Hz), 1.22 (m, 6H), 1.38 (s, 3H), 1.70 (m, 6H), 2.04 (m, 3H), 2.50 (m, 3H), 3.21 (m, 1H), 5.82 (s, 1H), 6.77 (d, 2H, J=8.17 Hz), 7.0 (d, 2H, J=8.23 Hz); ¹³C NMR (CDCl₃): δ13.02 (3×CH), 18.33 (6×CH₃), 20.20, 23.58, 25.25, 25.55, 26.49, 29.37, 33.97, 35.23, 36.31, 37.88, 38.04, 43.13, 50.46, 79.58, 93.77, 105.68, 120.06 (2C, Ar), 129.53 (2C, Ar), 134.70, 154.43, 171.81; IR (neat): 2917, 2860, 1736, 1515, 1462, 1266, 1101, 1001, 914, 882, 681; ESI MS m/z: Calcd. For C₃₂H₅₀O₆Si [M+H)⁺559.3455, found 559.5 [M+H]⁺, 1116.9 (2M+H]⁺.

(+)-Octahydro-3,6α-dimethyl-3,12-epoxy-9α-(3′-(3,5-bis-trifluoromethylphenyl) propyl)-12H-pyrano[4,3j]-1,2-benzodioxepin-10(3H)-one (35).

This analog was synthesized as reported previously. ³² Yield, 43%; [α]_{D}²⁶ +92.0 (CHCl₃, c 0.5). ¹H NMR (CDCl₃): δ1.0 (d, 3H, J=5.6 Hz), 1.13 (m, 1H), 1.46 (s, 3H), 1.47-1.52 (m, 4H), 1.74 (m, 3H), 1.83 (m, 3H), 1.98 (m, 1H), 2.05-2.18 (m, 3H), 2.39 (m 1H), 2.78 (t, 2H, J=7.6 Hz), 5.92 (s, 1H), 7.65 (s, 2H), 7.71 (s, 1H); ¹³C NMR (CDCl₃): δ19.83, 24.70, 25.40, 29.13, 31.57, 33.86, 33.95, 35.52, 35.90, 37.52, 43.12, 44.95, 50.51, 80.13, 93.80, 105.33, 119.96, 122.08, 124.79, 128.57, 131.37, 131.70, 144.51 (2C), 171.51; IR (neat) 2925, 2868, 1740, 1381, 1279, 1168, 1136, 1001, 890; HRMS (ESI) m/z: Calcd. for C₂₅H₂₈F₆O₅Na [M+Na]⁺545.1739, found: 545.1740 [M+Na]⁺, 561.1382 [M+K]⁺; Anal. (C₂₅H₂₈F₆O₅) C, H.

(+)-Octahydro-3,6α-dimethyl-3,12-epoxy-9β-(3′-(p-N,N-dimethylaminop henyl) propyl)-12H-pyrano[4,3j]-1,2-benzodioxepin-10(3H)-one (36). Yield, 30%; mp. 167-169° C.; [α]_{D}²⁶ +68.0 (CHCl₃, C 1.0); ¹H NMR (CDCl₃): δ0.98 (d, 3H, J=5.73 Hz), 1.03 (m, 1H), 1.38 (m, 4H), 1.44 (s, 3H), 1.59-1.80 (m, 6H), 2.01-2.20 (m, 3H), 2.41-2.70 (m, 3H), 2.90 (s, 6H), 3.21 (ddd, 1H, J=5.33, 1.7, 5.2 Hz), 5.83 (s, 1H), 6.69 (d, 2H, J=8.61 Hz), 7.05 (d, 2H, J=8.52 Hz); ¹³C NMR (CDCl₃): δ20.23, 23.65, 25.29, 25.59, 26.52, 29.44, 34.03, 35.02, 36.35, 37.96, 38.05, 41.32 (2C), 43.10, 50.54, 79.62, 93.80, 105.74, 113.45 (2C, Ar), 129.32 (2C, Ar), 130.58, 149.51, 171.93; IR (neat): 2921, 1748, 1613, 1523, 1180, 1119, 1033, 996, 804; HRMS (ESI) m/z: Calcd. for C₂₅H₃₅NO₅ [M+H]⁺430.2593, found 430.2587 [M+H]⁺, 452.2424 [M+Na]⁺; Anal. (C₂₅H₃₅NO₅) C, H, N. 38. (Hydrochloride) ¹H NMR (CDCl₃): δ0.98 (d, 3H, J=5.52 Hz), 1.04 (m, 1H), 1.36 (m, 2H), 1.42 (s, 3H), 1.70 (m, 1H), 1.75 (m, 6H), 1.98 (m, 4H), 2.40 (m, 1H), 2.67 (m, 2H), 3.14 (s, 6H), 3.18 (m, 1H), 5.83 (s, 1H), 7.29 (d, 2H, J=8.16 Hz), 7.64 (d, 2H, J=8.04 Hz); IR (neat): 2933, 1736, 1519, 1454, 1376, 1196, 1119, 1037, 1000.

(+)-Octabydro-3,6α-dimethyl-3,12-epoxy-9α-(3′-(p-N,N-dimethylaminophenyl) propyl)-12H-pyrano[4,3j]-1,2-benzodioxepin-10(3H)-one (37). Yield, 32%; mp. 145-147° C.; [α]_{D}²⁶ +88.0 (CHCl₃, c 0.5); ¹H NMR (CDCl₃): δ0.98 (d, 3H, J=5.76 Hz), 1.37-1.44 (m, 4H), 1.45 (s, 3H), 1.64-1.77 (m, 7H), 2.04-2.13 (m, 4H), 2.37 (m, 1H), 2.53 (m, 2H), 2.90 (s, 6H), 5.89 (s, 1H), 6.69 (d, 2H, J=8.64Hz), 7.06 (d, 2H, J=8.55 Hz); ¹³C NMR (CDCl₃): δ19.96, 24.76, 25.54, 30.12, 31.66, 34.0, 34.12, 34.84, 35.95, 37.56, 40.99 (NMe₂), 42.63, 45.06, 50.51, 80.39, 93.72, 105.27, 113.07 (2C, Ar), 128.98 (2C, Ar), 130.51, 149.06, 172.05; IR (neat): 2917, 2851, 1732, 1621, 1523, 1343, 1209, 1168, 1102; LCMS ESI m/z: Calcd. for C₂₅H₃₅NO₅ [M+H]⁺430, found 430 [M+H]⁺, 447 [M+NH₄]⁺; Anal. (C₂₅H₃₅NO₅. EtOAc); C, H, N.

(+)-Octahydro-3,6α-dimethyl-3,12-epoxy-9β-(3′-N(^{t}butyloxycarbonyli)-propyl)-12H-pyrano[4,3j]-1,2-benzodioxepin-10(3H)-one (45)

Yield, 31%; ¹H NMR (CDCl₃): δ1.0 (d, J=5.6 Hz, 3H), 1.09 (m, 1H), 1.44 (s, 3H), 1.28-1.43 (m, 14H), 1.60 (m, 2H), 1.79 (m, 3H), 2.05 (m, 3H), 2.43 (m, 1H), 3.17 (m, 3H), 4.66 (brs, 1H), 5.85 (s, 1H); ¹³C NMR (CDCl₃): δ19.82, 23.32, 24.23, 24.85, 25.17, 28.41, 33.55, 35.90, 37.54, 37.74, 43.21, 50.05, 79.20, 93.53, 105.41, 155.97, 171.49; IR (neat): 3338, 2941, 1732, 1679, 1523, 1274, 1164, 1115, 1033, 996.

(+)-Octahydro-3,6α-dimethyl-3,12-epoxy-9α-(3′-(N(′butyloxycarbonyl)-propyl)12H-pyrano[4,3j]-1,2-benzodioxepin (9α isomer of 47 as a representative example).

To a stirred solution of 44 (0.68 g, 1.6 mmol) in dry CH₂Cl₂ (5 mL) at −78° C. was added 1M Diisobutylaluminum hydride in CH₂CI₂ (2 mL, 1.2 equiv.), and the reaction mixture was stirred for 2b. It was then quenched with saturated NaHCO₃ (5 mL), diluted with CH₂Cl₂ (10 mL), and allowed to warm to room temperature. The mixture was then washed with 10% HCl/saturated NH₄Cl (1:15 v/v). The organic layer was dried over Na₂SO₄, filtered, and the combined organic solvents were concentrated under reduced pressure to afford a residue, which was chromatographed on a short silica gel column using CH₂Cl₂-EtOAc (3:1) solvent system to yield 0.583 g (86%) of the lactol as an isomeric mixture, which was used for the next reduction.

To a stirred solution of the lactol (46) (0.573 g, 1.34 mmol) in dry CH₂Cl₂ (4 mL) at −78° C. was added triethylsilane (1.07 mL, 6.7 mmol) and the mixture stirred for 10 min. To this was added BF₃.OEt₂ (0.255 mL, 2.01 mmol) and the mixture stirred at that temperature for 7 h. It was then quenched with pyridine (8 equiv., 0.87 mL), and allowed to warm to room temperature. This mixture was then poured into aqueous saturated NH₄Cl (10 mL) and extracted with EtOAc (3×25 mL). The combined organic layers were dried over anhydrous Na₂SO₄ and filtered. The solvents were evaporated in vacuo. The residue was chromatographed over silica gel using 15% EtOAc-hexanes to afford 0.25 g of the 10-deoxo derivative.

Yield, 45%; %; ¹H NMR (CDCl₃): δ0.95 (d, J=6 Hz, 3H), 1.08 (m, 1H), 1.24 (m, 3H), 1.41 (s, 3H), 1.43 (s, 9H), 1.44-1.76 (m, 8H), 1.87-2.02 (m, m, 3H), 2.31 (d,d,d, J=3.6, 3.2, 3.2 Hz, 1H), 3.12 (m, 2H), 3.8 (dq, J=4.4, 4.4, 4.0, 4.0 Hz, 2H), 4.61 (brs, 1H), 5.2 (s, 1H); ¹³ C NMR (CDCl₃): δ20.14, 24.63, 25.87, 28.28, 28.43, 30.42, 30.64, 34.26, 36.41, 37.15, 40.03, 40.64, 43.71, 52.15, 64.85, 79.0, 81.23, 92.10, 103.34, 155.99. IR (neat): 3366, 2925, 1715, 1527, 1217, 1172, 1102, 1053, 878, 824.

(+)-Octahydro-3,6α-dimethyl-3,12-epoxy-9β-(3′-(N-(5-dimethylamino naphthalene-1-sulfonic acid)-amido)-propyl)-12H-pyrano[4,3j]-1,2-benzodioxepin (48)

To an ice cooled and stirred solution of 47 (0.095 g, 0.23 mmol) was added HCl/ether (2M, 1.2 mL), and left stirred for 5 h for complete deprotection. The solvent was evaporated, and the residue re-dissolved in dry CH₂Cl₂. This was cooled in an ice bath, stirred and triethylamine (0.1 mL) was added to it followed by dansyl chloride (0.069 g, 0.253 mmol). The mixture was stirred for 24 h, washed with water (1×10 mL), extracted with EtOAc (3×15 mL), dried (MgSO₄), solvents evaporated under reduced pressure, and the residue chromatographed on a silica gel column using 15% EtOAc-hexanes to afford 0.078 g of the product. Yield, 63%, %; ¹H NMR (CDCl₃): δ0.92 (m, 2H), 0.94 (d, J=6 Hz, 3H), 1.13-1.38 (m, 10H), 1.40 (s, 3H), 1.59 (m, 1H), 1.83 (m, 1H), 2.02 (m, 1H), 2.22 (m, 1H), 2.33 (ddd, J=6, 3.6, 3.6 Hz, 1H), 2.91 (s, 6H), 3.23 (dd, J=12, 11.6 Hz, 1H), 3.55 (dd, J=2.8, 3.2 Hz, 1H), 4.91 (t, J=5.8 Hz, 1H, NH), 5.11 (s, 1H), 7.20 (d, J=7.6 Hz, 1H), 7.55 (m, 2H), 8.2 (dd, J=8.4, 7.2 Hz, 2H), 8.55 (d, J=8.4 Hz, 1H); ¹³C NMR (CDCl₃): δ20.28, 20.72, 24.66, 24.83, 26.06, 27.08, 32.73, 33.89, 36.18, 37.23, 43.24, 43.45, 45.46, 52.10, 53.48, 64.67, 80.53, 92.26, 104.16, 115.19, 118.71, 123.26, 128.43, 129.58, 129.69, 129.86, 130.48, 134.77, 152.01; IR (neat): 3300, 2933, 1695, 1450, 1319, 1143, 1094, 1061, 788.

(+)Octahydro-3,6α-dimethyl-3,12-epoxy-9β-(2′-cyanoethyl)-12H-pyranol[4,3j]-1,2-benzodioxepin-10(3H)-one -(52) Yield, 14%, mp. 171-172° C.; [α]_{D}²⁴ +120.0 (CHCl₃, c 0.5); ¹H NMR (CDCl₃): δ1.0 (d, 3H, J=5.55 Hz), 1.12 (m, 2H), 1.42 (m, 3H), 1.45 (s, 3H), 1.61-1.64 (m, 2H), 1.79-1.88 (m, 2H), 2.01-2.10 (m, 2H), 2.20-3.30 (m, 1H), 2.40-2.48 (m, 1H), 2.57-2.63 (m, 1H), 2.77-2.85 (m, 1H), 3.30 (m, 1H), 5.87 (s, 1H); ¹³C NMR (CDCl₃): δ16.37, 19.76, 23.60, 24.58, 24.78, 25.12, 33.35, 35.81, 37.38, 37.45, 44.36, 49.89, 79.05, 93.87, 105.60, 119.27, 170.64; IR (neat): 2933, 2864, 1732, 1442, 1384, 1115, 984; HRMS (ESI) m/z: Calcd. for C₁₇H₂₃NO₅Na [M+Na]⁺344.1474, found 344.1492 [M+Na]⁺; Anal. (C₁₇H₂₃NO₅)C, H.

(+)-Octahydro-3,6α-dimetbyl-3,12-epoxy-9β-([1,3]-dioxan-2-yl-ethyl)-12H-pyrano[4,3j]-1,2-benzodioxepin-10(3H)-one (53)

Yield, 17%; ¹H NMR (CDCl₃): δ0.98 (d, 3H, J=5.6 Hz), 1.06 (d, 2H), 1.29 (m, 1H), 1.41 (s, 3H), 1.34-1.48 (m, 3H), 1.65 (m, 1H), 1.81 (m, 5H), 1.97-2.16 (m, 4H), 2.39 (m, 1H), 3.23 (m, 1H), 3.83 (m, 2H), 3.94 (m, 2H), 4.86 (t, J=4.4 Hz, 1H), 5.83 (s, 1H); ¹³C NMR (CDCl₃): δ19.81, 21.15, 23.23, 24.84, 25.16, 31.21, 33.56, 35.89, 37.51, 37.58, 42.97, 50.05, 64.88, 64.94, 79.22, 93.45, 104.01, 105.34; IR (neat): 2929, 1732, 1454, 1217, 1139, 1111, 1033, 1008, 886.

(+)-Octahydro-3,6α-dimethyl-3,12-epoxy-9β-(3′-(p-methanesulfonylphenyl)propyl)-12H-pyrano-[4,3j]-1,2-benzodioxepin-10(3H)-one (30)

To a stirred solution of 28 (0.42 g, 0.96 mmol) in dry CH₂Cl₂ (40 mL) at −78° C. was added a solution of mCPBA (0.49 g, 77%, 2.2 mmol) in CH₂Cl₂ (40 mL) over a period of 2.5 h. The reaction mixture was stirred at that temperature for another 1.5 h. To this mixture was added water (10 mL), followed by solid NaHCO₃ (7.0 g). The reaction mixture was allowed to warm to room temperature, and the product was extracted with CH₂Cl₂ (3×30 mL). The combined organic layers were washed sequentially with 7% NaHCO₃ solution, water (1×25 mL), brine (1×25 mL), and dried over anhydrous Na₂SO₄. The solvents were removed under reduced pressure to afford the crude product, which was purified by flash column chromatography on silica gel using 3-4% EtOAc-CH₂Cl₂ as the eluent to yield 0.42 g of 30. Yield, 94%; mp. 157-158° C.; [α]_{D}²⁵ +59.0 (CHCI₃, c 1.0); ¹H NMR (CDCl₃): δ0.99 (d, 3H, J=5.46 Hz), 1.08 (m, 2H), 1.30 (m, 4H), 1.39 (s, 3H), 1.57 (d, 1H, J=4.7 Hz), 1.74 (m, 4H), 2.02 (m, 3H), 2.41 (m, 1H), 2.75 (m, 2H), 3.04 (s, 3H), 3.21 (m, 1H), 5.83 (s, 1H), 7.37 (d, 2H, J=8.07 Hz), 7.84 (d, 2H, J=8.03 Hz); ¹³C NMR (CDCl₃): δ14.47, 20.13, 22.96, 23.65, 25.16, 25.50, 26.78, 28.90, 31.90, 33.86, 35.98, 36.24, 37.78, 38.07, 43.46, 44.90, 50.37, 79.57, 93.85, 105.70, 127.82 (2C, Ar), 129.70 (2C, Ar), 138.54, 148.91, 171.66; IR (KBr): 3017, 2926, 2869, 1735, 1302, 1191, 1146, 1110, 998; ESI MS m/z: Calcd. for C₂₄H₃₂O₇S [M+H]⁺465.1947, found 465.2 [M+H]⁺, 929.3 (2M+H]⁺, 951.3 [2M+Na]⁺; Anal. (C₂₄H₃₂O₇S) C, H.

(+)-Octahydro-3,6α-dimethyl-3,12-epoxy-9β-(3′-(p-hydroxyphenyl)propyl)-12H-pyrano[4,3j]-1,2-benzodioxepin-10(3H)-one (33)

To a stirred solution of 31 (0.76 g, 1.36 mmol) in dry THF (12 mL) was added TBAF (0.34 mL, 1.0 M solution in THF) drop-wise and the mixture was stirred at room temperature for 2 h, monitoring the progress of the reaction by tlc. The solvent was evaporated under reduced pressure, and the residue purified by flash column chromatography on silica gel using 15% EtOAc-hexanes as the eluent to afford 0.5 g of 33. Yield, 91%; mp. 116-117° C.; [α]_{D}²⁵ +78.0 (CHCl₃, c 1.0); ¹H NMR (CDCl₃): δ0.98 (d, 3H, J=5.56 Hz), 1.03 (m, 2H), 1.34 (m, 4H), 1.43 (s, 3H), 1.45-1.79 (m, 6H), 2.05 (m, 3H), 2.40-2.70 (m, 3H), 3.21 (m, 1H), 5.83 (s, 1H), 6.74 (d, 2H, J=8.42 Hz), 7.03 (d, 2H, J=8.31 Hz); ¹³C NMR (CDCl₃): δ20.19, 23.64, 25.24, 25.54, 26.51, 29.46, 33.94, 35.16, 36.31, 37.89, 38.14, 43.12, 50.43, 79.68, 94.03, 105.86, 115.74 (2C, Ar), 129.70 (2C, Ar), 133.92, 154.57, 172.60; IR (KBr): 3386, 2928, 2872, 1733, 1715, 1613, 1514, 1445, 1378, 1202, 1113, 1000; ESI MS (m/z) calcd for C₂₃H₃₀O₆ [M+H]⁺403.2, found 403.2 [M+H]⁺, 805.6 (2M+H]⁺, 827.5 [2M+Na]⁺; Anal. (C₂₃H₃₀O₆. 0.5 H₂O) C, H.

(+)-Octahydro-3,6α-dimetbyl-3,12-epoxy-9β-(3′-(p-N,N-dimethylaminophenyl)propyl)-12H-pyrano-[14,3j]-1,2-benzodioxepin (43).

To a stirred solution of 36 (0.41 g, 0.953 mmol) in dry CH₂Cl₂ (5 mL) at −78° C. was added 1M Diisobutylaluminum hydride in CH₂Cl₂ (1.1 mL, 1.1 equiv.), and the reaction mixture was stirred for 2h. It was then quenched with saturated NaHCO₃ (5 mL), diluted with CH₂Cl₂ (10 mL), and allowed to warm to room temperature. The mixture was then washed with 10% HCl/saturated NH₄Cl (1:15 v/v). The organic layer was dried over Na₂SO₄, filtered, and the combined organic solvents were concentrated under reduced pressure to afford a residue, which was chromatographed on a short silica gel column using CH₂Cl₂-EtOAc (3:1) solvent system to yield 0.333 g (82%) of the lactol as an isomeric mixture, which was used for the next reduction.

To a stirred solution of the lactol (42) (0.33 g, 0.77 mmol) in dry CH₂Cl₂ (6 mL) at −78° C. was added triethylsilane (0.49 mL, 3.06 mmol) and the mixture stirred for 10 min. To this was added BF₃.OEt₂ (0.5 mL, 1.15 mmol) and the mixture stirred at that temperature for 7 h. It was then quenched with pyridine and allowed to warm to room temperature. This mixture was then poured into aqueous saturated NH₄Cl (10 mL) and extracted with EtOAc (3×25 mL). The combined organic layers were dried over anhydrous Na₂SO₄ and filtered. The solvents were evaporated in vacuo. The residue was chromatographed over silica gel using 15% EtOAc-hexanes to afford 0.11 g of 43. Yield, 35%; mp. 131-132° C.; [α]_{D}²⁶ +78.0 (CHCl₃, c 0.5); ¹H NMR (CDCl₃): δ0.95 (d, 3H, J=6.13 Hz), 0.96-1.40 (m, 4H), 1.42 (s, 3H), 1.44-1.74 (m, 8H), 1.80-1.95 (m, 1H), 1.96-2.10 (m, 1H), 2.30-2.60(m, 4H), 2.91 (s, 6H), 3.42 (dd, 1H, J=11.69, 11.70 Hz), 3.79 (dd, 1H, J=3.78, 3.95 Hz), 5.19 (s, 1H), 6.68 (d, 2H, J=8.53 Hz), 7.03 (d, 2H, J=8.52 Hz); ¹³C NMR (CDCl₃): δ20.71, 21.31, 25.16, 26.52, 27.63, 29.02, 33.34, 34.47, 35.25, 36.66, 37.74, 41.33 (2C, NMe₂), 43.67, 52.65, 65.66, 81.10, 92.82, 104.56, 113.40 (2C, Ar), 129.30 (2C, Ar), 130.78, 150.0; IR (neat): 2933, 1621, 1523, 1454, 1348, 1135, 1098, 1061, 874, 808 HRMS (ESI) m/z: Calcd. for C₂₅H₃₇NO₄ [M+H]⁺416.2801, found 416.2812 [M+H]⁺, 438.2639 [M+Na]⁺; Anal. (C₂₅H₃₇NO₄); C, H, N. 43a (hydrochloride). 0.89 (d, 3H, J=6.12 Hz), 0.98-1.29 (m, 3H), 1.35 (s, 3H), 1.38-1.66 (m, 9H), 1.80 (m, 1H), 1.84-1.97 (m, 1H), 2.27 (dd, 1H, J=3.20, 3.70 Hz), 2.38 (m, 1H), 2.55 (t, 2H, J=7.23 Hz), 3.12 (s, 6H), 3.39 (dd, 1H, J=5.83, 11.64 Hz), 3.70 (dd, 1H, J=3.48, 3.57 Hz), 5.13 (s, 1H), 7.22 (d, 2H, J=7.7 Hz), 7.63 (d, 2H, J=7.86. Hz); ¹³C NMR (CDCl₃): δ20.65, 21.28, 25.05, 26.44, 27.50, 28.56, 33.26, 34.33, 35.69, 36.58, 37.64, 43.70, 47.03, 52.55, 53.88, 65.34, 81.00, 92.71, 104.55, 121.02 (2C, Ar), 130.66 (2C, Ar), 141.10, 144.99, 162.73.

Preparation of C-9 substituted thioether 55

To a stirred solution of artemisitene (0.1 g, 0.36 mmol) in methanol at room temperature was added Boc-Cysteine-CO₂Me (0.093 g, 0.392 mmol) and the mixture stirred for 4 h. The solvent was evaporated under reduced pressure and the residue chromatographed on a silica gel column using 15% EtOAc-hexanes to afford 0.068 g of the product 55. Yield 61%; ¹H NMR (CDCl₃): δ1.01 (d, J=6 Hz, 3H), 1.27 (m, 2H), 1.45 (s, 3H), 1.46, (s, 9H), 1.48 (m, 3H), 1.74 (m, 2H), 1.97 (m, 1H), 2.10 (m, 2H), 2.27 (m, 1H), 2.39 (m, 1H), 2.86 (dd, J=12.8, 11.2 Hz, 1H), 3.02 (m, 2H), 3.30 (dd, J=3.6, 4.0 Hz, 1H), 3.80 (s, 3H), 4.56 (brs, 1H), 5.40 (d, J=8 Hz, 1H), 5.95 (s, 1H); ¹³C NMR (CDCl₃): δ19.86, 24.73, 25.45, 28.30, 31.45, 33.85, 34.73, 35.85, 37.34, 37.60, 41.20, 44.51, 50.33, 52.75, 53.18, 80.23, 80.60, 94.08, 105.42, 155.05, 170.2, 171.29; IR (neat): 3382, 2933, 1711, 1499, 1364, 1217, 1168, 1102, 1041, 759.

Preparation of C-10 substituted thioether 59

To a stirred solution of dihydroartemisinin³⁴ (0.5 g, 1.76 mmol) in dry CH₂Cl₂ at −78° C. was added Boc Cysteine-CO2Me, followed by BF₃.OEt₂ dropwise. The mixture was left stirred for 5 h, and poured in to a saturated solution of NH₄Cl (10 mL). It was then extracted with EtOAc (3×20 mL), organic layers combined, dried (MgSO₄), and the solvents evaporated under reduced pressure to afford a residue, which was purified by chromatography on a silica gel column to yield 0.12 g of the product as gummy liquid. Yield, 24% (based on artemisinin recovered); ¹H NMR (CDCl₃): δ0.96 (d,d, J=6 Hz, 7.2 Hz, 6H), 1.46 (s, 12H), 1.38-1.60 (m, 5H),1.71 (m, 3H), 1.88 (m, 1H), 2.08 (m, 1H), 2.38 (m, 1H), 2.95 (m, 1H), 3.06 (m, 1H), 3.30 (m, 1H), 3.77 (s, 3H), 4.73 (brs, 1H), 5.22 (d, J=4.8 Hz, 1H), 5.60 (s, 1H), 6.04 (d, J=9.6 Hz, 1H, NH); ¹³C NMR (CDCl₃): δ14.90, 20.27, 24.35, 25.54, 26.11, 28.35, 28.36, 32.18, 34.36, 36.27, 37.20, 45.00, 52.33, 52.55, 53.95, 79.81, 80.95, 88.02, 88.36, 104.29, 155.64, 171.43; IR (neat): 3330, 2937, 1752, 1715, 1515, 1368, 1172, 1029, 931.

Preparation of 1-deoxo analogs 117-121.

A solution of the appropriate artemisinin analog (0.35 mmol) in ethyl acetate (6 mL) was mixed with palladium on charcoal (0.02 g 10% w/w) and hydrogenolyzed under a positive pressure of hydrogen for 7h, monitoring the progress of reaction by tlc. The reaction mixture was filtered, the solvents evaporated under reduced pressure and the product purified by chromatography on silica gel column, eluting with 10% ethyl acetate-hexanes to afford the corresponding 1-deoxo compounds.

(+)-Octahydro-3,6α,9β-trimethyl-3,12-epoxy-12H-pyrano[4,3j]-1,2-benzoxepin-10(3H)-one (117). Prepared from artemisinin 1. Yield, 54%; mp. 107-108° C.; [α]_{D}²⁶-75.0 (CHCl₃, c 1.0); ¹H NMR (CDCl₃): δ0.94 (d, 3H, J=5.33 Hz), 1.01 (m, 1H), 1.19 (d, 3H, J=7.21 Hz), 1.25 (m, 4H), 1.52 (s, 3H), 1.59 (m, 1H), 1.76 (m, 2H), 1.92 (m, 2H), 2.0 (m, 1H), 3.19 (m, 1H), 5.69 (s, 1H); 13C NMR (CDCl₃): δ12.64, 18.60, 22.02, 23.53, 23.99, 32.76, 33.45, 33.97, 35.36, 42.41, 44.61, 82.42, 99.65, 109.23, 171.89; IR (neat), 2941, 1748, 1384, 1139, 1009, 1001, 874; HRMS (ESI) m/z: Calcd. for C₁₅H₂₂O₄Na [M+Na)⁺289.1416, found 289.1424 [M+Na]⁺, 305.1171 [M+K]⁺; Anal. (C₁₅H₂₂O₄) C, H.

(+)-Octabydro3,6α-dimethyl-3,12-epoxy-9β-(3′-(3,5-bis-trifluoromethylphenyl) propyl)-12H-pyrano[4,3j]-1,2-benzoxepin-10(3H)-one (118) Prepared from 34. Yield, 21%; [α]_{D}²⁹ −32.0 (CHCl₃, c 1.0); ¹H NMR (CDCl₃): δ0.93 (d, 3H, J=4.97 Hz), 1.05 (m, 1H), 1.27 (m, 4H), 1.53 (s, 3H), 1.55-1.70 (m, 2H), 1.79 (m, 4H), 1.91 (m, 2H), 2.05 (m, 2H), 2.77 (m, 2H), 3.01 (m, 1H), 5.68 (s, 1H), 7.64 (s, 2H), 7.71 (s, 1H); ¹³C NMR (CDCl₃): δ18.55, 22.01, 23.59, 23.96, 26.75, 28.80, 33.39, 33.97, 35.34, 35.57, 37.68, 40.46, 44.65, 54.34, 82.17, 99.31, 109.34, 120.04 (2C), 122.06, 128.49 (2C), 131.43, 131.75, 144.37, 171.04; IR (neat): 2921, 1744, 1384, 1287, 1164, 1127, 1013, 894; HRMS (ESI) m/z: Calcd. for C₂₅H₂₈F₆O₄Na [M+Na]⁺529.1789, found: 529.1769 [M+Na]⁺, 545.1514 [M+K]⁺.

(+)-Octahydro3,6α-dimetbyl-3,12-epoxy-9β-(3′-p-methanesulfonylphenyl) propyl)-12H-pyrano[4,3j]-1,2-benzoxepin-10(3H)one (119). Prepared from 30. Yield, 32%; mp. 160-161° C.; [α]_{D}²⁵ +38.0 (CHCl₃, c 0.5); ¹H NMR (CDCl₃): δ0.94 (d, 3H, J=4.4 Hz), 1.07 (m, 1H), 1.27-1.36 (m, 4H), 1.53 (s, 3H), 1.59-1.69 (m, 2H), 1.78 (m, 4H), 1.85-1.93 (m, 2H), 2.04 (m, 2H), 2.75 (m, 2H), 2.99 (ddd, 1H, J=4.4, 4.4, 4.4 Hz), 3.05 (s, 3H), 5.68 (s, 1H), 7.40 (d, 2H, J=8.0 Hz), 7.86 (d, 2H, J=8.4 Hz); ¹³C NMR (CDCl₃): 18.56, 21.99, 23.57, 23.97, 26.62, 28.73, 33.39, 33.95, 35.32, 35.77, 37.69, 40.38, 44.60, 44.65, 82.16, 99.28, 109.30, 127.51 (2C, Ar), 129.36 (2C, Ar), 138.14, 148.63, 171.14; IR (neat), 2929, 1748, 1392, 1307, 1143, 1106, 1029, 869; HRMS (ESI) m/z: Calcd. for C₂₄H₃₂O₆SNa [M+Na]⁺471.1817, found: 471.1790 [M+Na]⁺, 487.1535 [M+K]⁺; Anal. (C₂₄H₃₂O₆S. 0.5 EtOAc) C, H.

(+)-Octahydro-3,6α-dimethyl-3,12-epoxy-9β-(3′-(p-trifluoromethyl phenyl) propyl)-12H-pyrano[4,3j]-1,2-benzoxepin (120). Prepared from 98. Yield, 42%; mp. 82-83° C.; [α]_{D}²⁶ −7.0 (CHCl₃, c 1.0); ¹H NMR (CDCl₃): δ0.91 (d, 3H, J=5.2 Hz), 1.02 (m, 1H), 1.17-1.39 (m, 6H), 1.54 (s, 3H), 1.57-1.74 (m, 6H), 1.85 (m, 1H), 1.94 (m, 1H), 2.13 (m, 1H), 2.68 (t, 2H, J=7.2 Hz), 3.37 (dd, H, J=4.4, 4.4 Hz), 3.94 (dd, 1H, J=7.2, 7.2 Hz), 5.27 (s, 1H), 7.29 (d, 2H, J=7.6 Hz), 7.54 (d, 2H, J=8.0 Hz); ¹³C NMR (CDCl₃): δ18.85, 22.12, 23.88, 24.01, 29.02, 30.68, 31.65, 34.43, 34.46, 35.41, 35.87, 39.16, 45.77, 62.83, 82.38, 96.43, 107.34, 123.02, 125.22, 125.72, 127.98, 128.30, 128.66, 146.43; IR (neat), 2941, 1323, 1160, 1127, 1066, 1001, 886; HRMS (ESI) m/z: Calcd. for C₂₄H₃₁F₃O₃Na [M+Na]⁺447.2123, found: 447.2113 [M+Na]⁺, 463.1857 [M+K]⁺; Anal. (C₂₄H₃₁F₃O₃) C, H.

(+)-Octahydro-3,6α-dimethyl-3,12-epoxy-9β-(3′-(m-chlorophenyl)propyl)-12H-pyranol4,3j]-1,2-benzoxepin (121). Prepared from 96. Yield, 47%; [α]_{D}²⁶ +30.0 (CHCl₃, c 0.5); ¹H NMR (CDCl₃): δ0.92 (d, 3H, J=5.6 Hz), 1.03 (m, 1H), 1.17-1.39 (m, 7H), 1.55 (s, 3H), 1.59-1.64 (m, 2H), 1.71 (m, 3H), 1.85 (m, 1H), 1.95 (m, 1H), 2.13 (m, 1H), 2.60 (t, 2H, J=7.2 Hz), 3.37 (dd, 1H, J=4.4, 4.8 Hz), 3.94 (dd, 1H, J=7.2, 6.8 Hz), 5.27 (s, 1H), 7.06 (d, 1H, J=6.8 Hz), 7.20 (m, 3H); ¹³C NMR (CDCl₃): δ18.87, 22.14, 23.87, 24.03, 29.05, 30.68, 31.66, 34.44, 34.47, 35.43, 35.72, 39.14, 45.78, 62.90, 82.40, 96.44, 107.34, 125.96, 126.58, 128.48, 129.56, 134.05, 144.37; IR (neat) 2933, 1724, 1467, 1279, 1123, 1107, 1074, 1001, 882; IRMS (ESI) m/z: Calcd. for C₂₃H₃₁ClO₃K [M+K]⁺429.1599, found: 413.1873 [M+Na]⁺, 429.1596 [M+K])⁺; Anal. (C₂₃H₃₁ClO₃) C, H.

While the present invention has been described with reference to the specific embodiments thereof, it should be understood by those skilled in the art that various changes may be made and equivalents may be substituted without departing from the true spirit and scope of the Invention. In addition, many modifications may be made to adapt a particular situation, material, composition of matter, process, process step or steps, to the objective, spirit and scope of the present invention. All such modifications are intended to be within the scope of the claims appended hereto.

### LIST OF REFERENCES

(1) Desowitz, R. S. The Malaria Capers (More Tales of People, Research and Reality; W. W. Norton & Company: New York, 1991.
(2) Van Agtmael, M. A.; Eggelte, T. A.; Van Boxtel, C. J. Trends Pharmacol. Sci. 1999, 20, 199-205.(6)
(3) Hien, T. T. W., N. J. The Lancet 1993, 341, 603-608.(2)
(4) Luo, X. D.; Shen, C. C. Med. Res. Rev. 1987, 7, 29-52.(3)
(5) People's Health Publishing,: Lanzhou, China, 1991.(4)
(6) Kem, P. Leishmaniasis. Antibiotics Chemother. 1981, 30, 203-223. (34*)
(7) De Carvalho, Paulo Batista; Anibas, Marco Aurelio Da Graca; Ferreira, Elizabeth Igne., Rev. Bras. Cienc. Farm. Leishmaniasis. What do we know about its chemotherapy? 2000, 36, 1, 69-96.
(8) Loiseau, P. M.; Bories, C. Recent strategies for the chemotherapy of visceral leishmaniasis. Curr. Opin. Infect. Dis. 1999, 12, 6, 559-564.
(9) Selzer, P. M.; Pingel, S.; Hsieh, I.; Ugele, B.; Chan, V. J.; Engel, J. C.; Bogyo, M.; Russell, D. G.; Sakanari, J. A.; McKerrow, J. H. Cysteine protease inhibitors as chemotherapy: lessons from a parasite target. Proc. Natl. Acad. Sci. U. S. A. 1999, 96, 20, 11015-11022.
(10) Moreno, J.; Canavate, C.; Chamizo, C.; Laguna, F.; Alvar, J. HIV-Leishmania infantum co-infection: humoral and cellular immune responses to the parasite after chemotherapy. Trans R. Soc. Trop. Med. Hyg. 2000, 94, 3, 328-332.
(11) Kennedy M L; Cortes-Selva F; Perez-Victoria J M; Jimenez I A; Gonzalez A G; Munoz O M; Gamarro F; Castanys S; Ravelo A G. Chemosensitization of a multidrug-resistant Leishmania tropica line by new sesquiterpenes from Maytenus magellanica and Maytenus chubutensis. J. Med. Chem., 2001, 44, 4668-4676.
(12) Bennan, J. D., and Grog, 1. M. Leishmania mexicana: Chemistry and biochemistry of sodium stibogluconate (Pentostam). Experimental Parasitology, 1988, 67, 96-103.(40)
(13) Brost, P., and Ouellette, M. New mechanisms of drug resistance in parasitic Protozoa. Ann. Rev. Microbiol., 1995, 49, 427-460.(41)
(14) Olliaro, P. L., and Bryceson, A. D. M. Practical progress and new drugs for changing patterns of leishmaniasis. Parasitology Today, 1993, 9, 323-8.(42).
(15) Thakur, C. P. Comparison of glucose versus fat emulsion in the preparation of amphotericin B for use in kala-azar. Transactions of the Royal Society for tropical medicine and Hygiene, 1994, 88, 689-699.(43)
(16) Dietz, R.; Fagundes, S. M. S.; Brito, E. F.; Milan, E. P.; Fietosa, T. F.; Suassuna, F. A. B.; Fonschiffrey, G.; Ksionski, G., and Dember, J. Treatment of kalα-azar in Brazil with Amphocil (amphotericin B cholesterol dispersion) for 5 days. Transactions of the Royal Society for tropical medicine and Hygiene, 1995, 89, 309-311.(44)
(17) The necessity to develop drugs against parasitic diseases., Kaiser, A.; Gotrwald, A.; Wiersch, C.; Maier, W.; Seitz, H. M. Pharmazie, 2002, 57, 11, 723-728.
(18) Recent developments in human African trypanosomiasis. Welbum, Susan C.;

Odiit, Martin. Current Opinion in Infectious Diseases, 2002, 15, 5, 477-484.
(19) Developments in the treatment of leishmaniasis and trypanosomiasis. Olliaro, Piero; Lazdins, Janis; Guhl, Felipe, Expert Opinion on Emerging Drugs. 2002, 7, 1, 61-67.
(20) Chemotherapy of human African trypanosomiasis. Burchmore, Richard J. S.;

Ogbunude, Patrick 0. J.; Enanga, Bertin; Barrett, Michael P., Current Pharmaceutical Design, 2002, 8, 4, 257-267.
(21) Klayman, D. L. Science (Washington, D. C., 1883-) 1985, 228, 1049-55.(8)
(22) Van Geldre, E.; Vergauwe, A.; Van den Eeckhout, E. Plant Mol. Biol. 1997, 33, 199-209.(9)
(23) Vroman, J. A.; Alvim-Gaston, M.; Avery, M. A. Curr. Pharm. Des. 1999, 5, 101-138.(10)
(24) Brossi, A.; Venugopalan, B.; Dominguez Gerpe, L.; Yeh, H. J. C.; Flippen-Anderson, J. L.; Buchs, P.; Luo, X. D.; Milhous, W.; Peters, W. J. Med. Chem. 1988, 31, 645-50.(1l)
(25) Brewer, T. G.; Grate, S. J.; Peggins, J. O.; Weina, P. J.; Petras, J. M.; Levine, B.

S.; Heiffer, M. H.; Schuster, B. G. In Am. J. Trop. Med. Hyg. 1994; Vol. 51, p 251-9.(12)
(26) Brewer, T. G.; Peggins, J. O.; Grate, S. J.; Petras, J. M.; Levine, B. S.; Weina, P.

J.; Swearengen, J.; Heiffer, M. H.; Schuster, B. G. Trans. R. Soc. Trop. Med. Hyg. 1994, 88, 33-36.(13)
(27) Haynes, R. K.; Hung-On, P. H.; Voerste, A., Ring opening of artemisinin (qinghaosu) and dihydroartemisinin and interception of the open hydroperoxides with formation of N-oxides—a chemical model for antimalarial action. Tet. Lett., 1999, 40, 25, 4715-4718.(12b*)
(28) Makler, M. T., Ries J. M., Williams J. A., Bancroft J. E., Piper R. C., Gibbins B. L. and Hinriches D. J. (1993) Parasite lactate dehydrogenase as an assay for Plasmodium falciparum drug sensitivity, Am. J. Trop. Med. Hyg., 48, 739-741.(47)
(29) a) Sereno, Denis; Roy, Gaetan; Lemesre, Jean Loup; Papadopoulou, Barbara;

Ouellette, Marc. DNA transformation of Leishmania infantum axenic amastigotes and their use in drug screening. Antimicrobial Agents and Chemotherapy, 2001, 45, 4, 1168-1173. b) Babich H. and Borenfreund, E. Cytotoxicity of T2 toxin and its metabolites with the neutral red cell viability assay, App. Envt Microbiol., 1991, 57,2101-2103.(48)
(30) Avery, M. A.; Maria Alvim-Gaston, and Woolfrey, J. R. Synthesis And Structure-Activity Relationships of Peroxidic Antimalarials Based on Artemisinin. Advances in Medicinal Chemistry, 1999, 4, 125-217.
(31) Avery, M. A.; Alvim-Gaston, M.; Rodrigues, C. R.; Barreiro, E. J.; Cohen, F. E.;

Sabnis, Y. A., and Woolfrey, J. R. Structure-Activity Relationships of the Antimalarial Agent Artemisinin. 6. The Development of Predictive In Vitro Potency Models Using CoMFA and HQSAR Methodologies. J. Med. Chem., 2002, 45, 292-303.
(32) Avery, M, A.; Alvim-Gaston, M.; Vroman, J. A.; Wu, B.; Ager, A.; Peters, W.;

Robinson, B. L.; Charman, W. Structure-Activity Relationships of the Antimalarial Agent Artemisinin. 7. Direct Modification of (+)-Artemisinin and In Vivo Antimalarial Screening of New, Potential Preclinical Antimalarial Candidates. J. Med. Chem., 2002, 45, 19, 4321-4335.
(33) Pal, J. K, and Purandare, M, J. Dose-dependent differential effect of hemin on protein synthesis and cell proliferation in Leishmania donovani promastigots cultures in vitro. J. Biosci. (Indian Acad. of Sciences), 2001, 26, 2, 225-231.
(34) Arteether, a New Antimalarial Drug: Synthesis and Antimalarial Properties.

Brossi, A.; Venugopalan, B.; Gerpe, D.; Yeh, H. J. C.; Anderson, J. L. F.; Buchs, P., and Luo, X. D., J. Med. Chem., 1988, 31, 645-650.

1. A method for the preparation of compounds of Formula 11 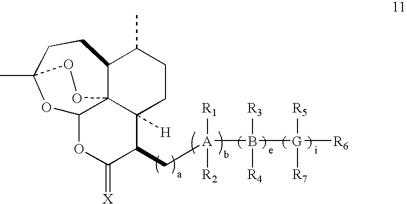
where X, A, B, G, a, b, e, i and R₁-R₇ are defined as follows:
X═O or H,OH (R or S) or H,OR (R orS) or H,H or H,NR (R or S); H,NRR′ (R or S); or H, SR (R and S) either as chiral, diastereomenrc, or racemic compounds A, B, G, E, J, L═C, CH, CH₂, C═O, CHOR (R or S); N, O, S wherein only chemically stable linkages are claimed (e.g., E═O, J═O is an acceptable peroxide if L contains stabilizing substituents at R₁₂ and/or R₁₃); A could also come from condensation with 1,3-dicarbonyls such that R_{1,2}=COMe or COOEt or combinations thereof;
with the proviso that if A, B or G are C, C=0, N, O, S; certain of the substituents are nonexistent.
b=0,1; e=0,1; i=0,1;a=1-6
R, R′, R₁-R₇=H or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO₂R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂)ₙNRR′; phenol, alkoxy (R′O), aryloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, furanyl, thiopheneyl, and benzo homologues (e.g. indolyl, quinolyl, isoquinolyl, etc.); and substituted variants therein, wherein any A, B, G that is potentially chiral can be a diastereomer or enantiomer, or diastereomeric mixtures and racemates; said method comprising the following Scheme 1:

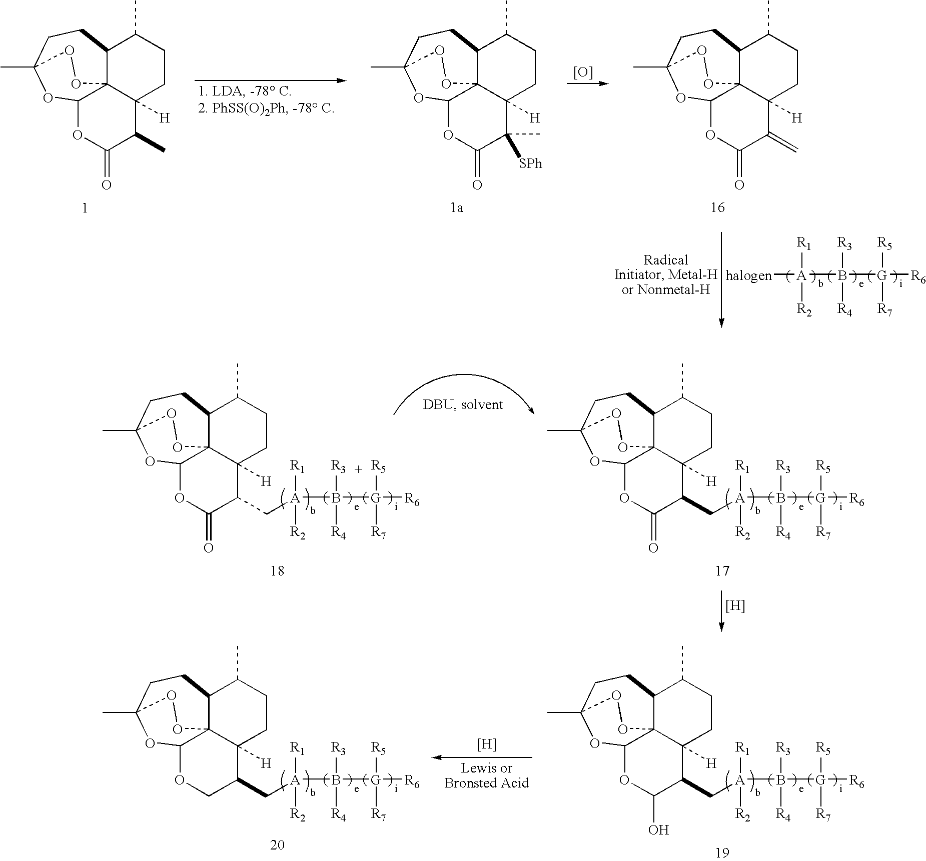

2. A method for the synthesis of compounds of the Formula 12 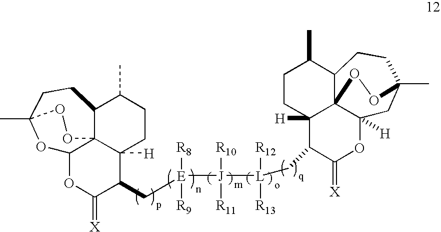
where X, E, J, L, p, q, n, m, o, and R₈-R₁₃ are defined as follows:
X═O or H,OH (R or S) or H,OR (R or S) or H,H or H,NR (R or S); H,NRR′ (R or S); E, J, L=CH₂, C═O, CHOR (R or S); N, O, S wherein only chemically stable linkages are claimed (e.g., E═O,J═O is an acceptable peroxide if L contains stabilizing substituents at R₁₂ and/or R₁₃). E or L could also come from condensation with 1,3-dicarbonyls such that R_{8,9} and or R_{12,13}═COMe or COOEt or combinations thereof;
n=0,1; m=0,1; o=0,1; p,q=1-6
R, R₈-R₁₃═H or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO₂R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂)ₙNRR′; phenol, alkoxy (R′O), aryloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, furanyl, thiopheneyl, and benzo homologues (e.g. indolyl, quinoly, isoquinolyl, etc.); and substituted variants therein., wherein any A, B. G that is potentially chiral can be a diastereomer or enantiomer, or diastereomeric mixtures and racemates; said method comprising the following Scheme 2 with the proviso that if A,B,G,E,J, X, V or L are C, C═O, N, O, or S, certain of the substituents are nonexistent:

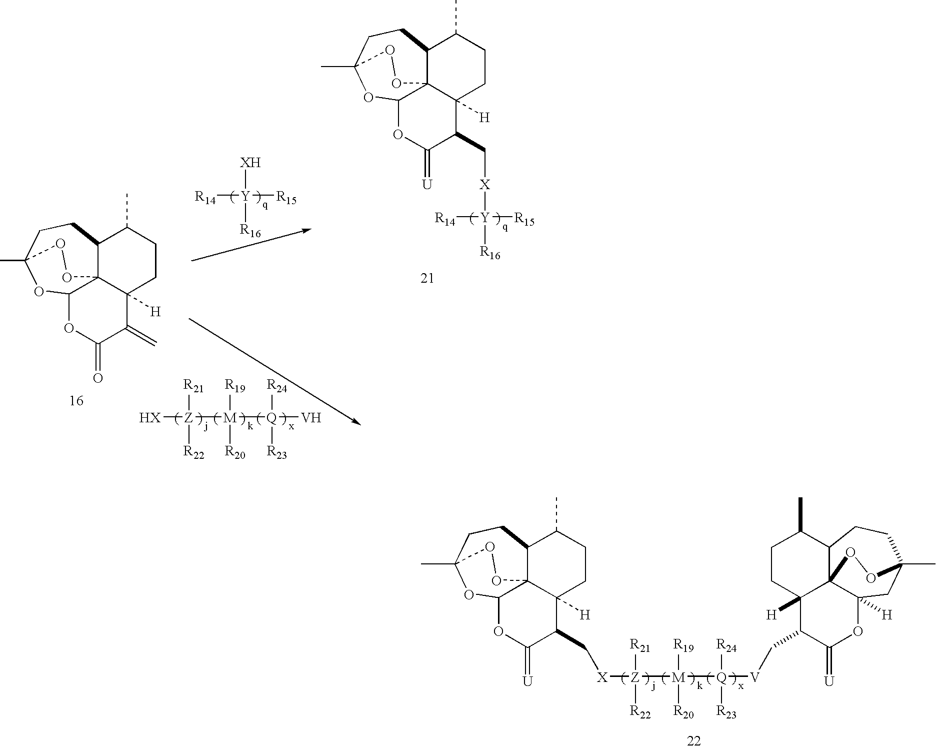

3. A method for the synthesis of compounds of the Formula 13 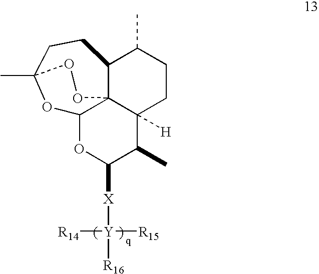
where X, Y and R₁₄-R₁₆ are defined as follows:
X═S, SO, SO₂, O, NH, NR, NOH, NOR, C═C, C═O, CH₂, Y═C, N, O, S, C═O, or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include, but are not limited to, halogen; thiol, thioalkyl, sulfonylalkyl (SOR′), sulfone (SO₂R′), sulfonamido (NHSO₂R′), acylsulfonamido (R″CONSO₂R′), N-alkylsulfonarnido (R″NSO₂R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂)ₙNRR′; phenol, alkoxy (R′O), aryloxy (ArO), haloalkyl; alkyl, alkenyl, arylalkyl, aiylalkenyl, aryl; substituted aryl; heteroaryl; substituted heteroaryl; R is also heterocyclic and heteroaromatic, such as pyridyl, pyrrolyl, fuiranyl, thiopheneyl, and benzo homologues (e.g. indolyl, quinolyl, isoquinolyl, etc.); and substituted variants therein;
q=0, 1, 2 etc.
R, R₁₄-R₁₆=H or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include, but are not limited to, halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO₂R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂)ₙNRR′; phenol, alkoxy (R′O), aryloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, furanyl, thiopheneyl, and benzo homologues (e.g. indolyl, quinolyl, isoquinolyl, etc.); and substituted variants therein and so on wherein any X and/or Y that is potentially chiral can be a diastereomer or enantiomer, or diastereomenrc mixtures and racemates with the proviso that if X,Y,Z,X′,M, or Q are C, C═O, N, O, or S, certain of the substituents are nonexistent; wherein the method comprises the following Scheme 3:

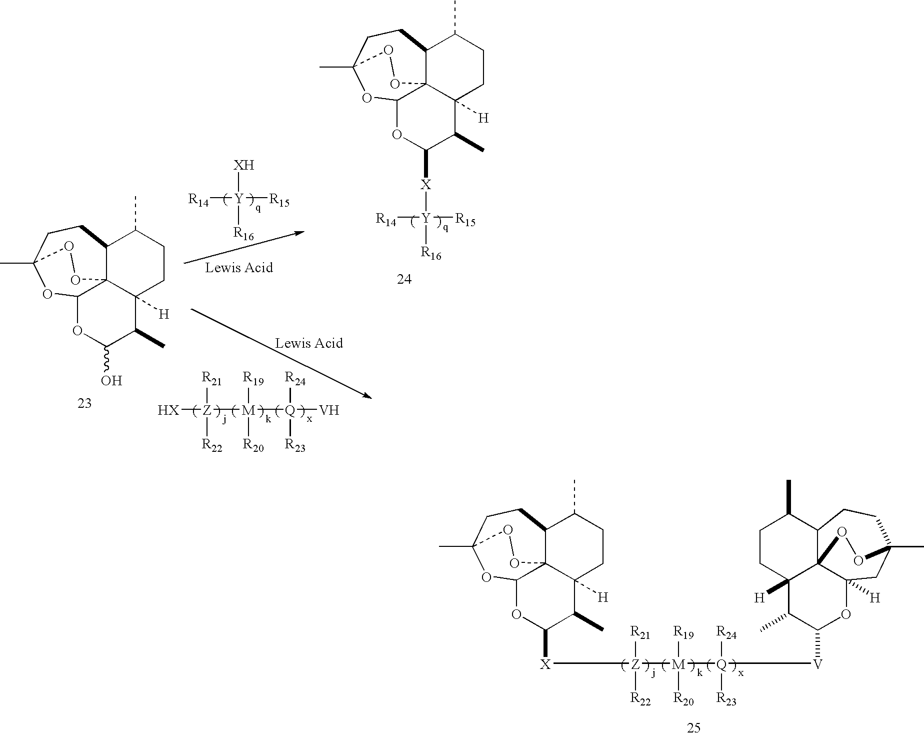

4. A method for the synthesis of compounds of the Formula 14. 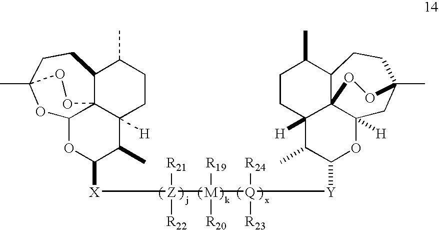
where X, Y, Z, M, Q, and R₂₁-R₂₄ are defined as follows:
X═S, SO, SO₂, O, C═O, CH₂,
Y═S, SO, SO₂, O, C═O, CH₂,
Z, M, Q═C, N, O, S, C═O, or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include, but are not limited to, halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO2R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂),NRR′; phenol, alkoxy (R′O), axyloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, flranyl, thiopheneyl, and benzo homologues (e.g. indolyl, quinolyl, isoquinolyl, etc.); and substituted variants therein;
q=0, 1, 2 etc.
R, R₁₄-R₁₆═H or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include, but are not limited to, halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO₂R′), nitro, amino, N-alkyl or N-aryl or N-heteroaryl amino; N,N-dialkylamiino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂)ₙNRR′; phenol, alkoxy (R′O), aryloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, furanyl, thiopheneyl, and benzo homologues (e.g. indolyl, quinolyl, isoquinolyl, etc.); and substituted variants therein and so on wherein any X, Y, Z, M and/or Q that is potentially chiral can be a diastereomer or enantiomer, or diastereomeric mixtures and racemates with the proviso that if X,Y,Z,X′,M, or Q are C, C═O, N, O, or S, certain of the substituents are nonexistent; wherein the method comprises the Scheme 4:

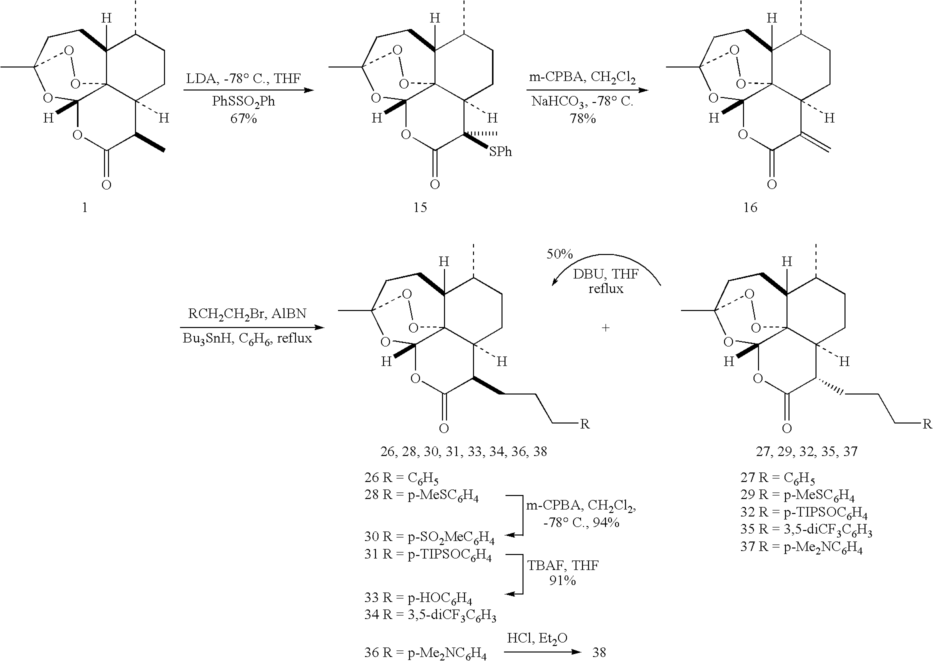

5. A method for the treatment of drug resistant or sensitive strains of infectious diseases such as, malaria (Plasmodium falciparum or other Plasmodia species), Leishmania (L. donovani, L. major and related species), Babesia (e.g. Babesia divergens), Toxoplasma gondii, HIV (human immunodeficiency virus), Tuberculosis, Schistosomiasis (e.g. Shistosoma mansonii or japonica), Trypanosoma cruzi (Chaga's desease), Trypanosoma brucei (African sleeping sickness) or diseases and conditions susceptible to oxidative damage such as acne vularis and other skin infections comprising administering to a subject in need of such treatment an effective amount of at least one compound prepared by the methods of claims 1, 2, 3 or 4.

6. A method for the treatment of various cancers and diseases of disrupted proliferation of tissues comprising administering to a subject in need of such treatment an effective amount of at least one compound prepared by the methods of claims 1, 2, 3 or 4.

7. A compound of the formula 11 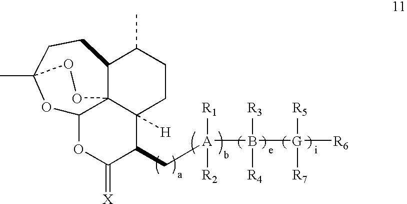
wherein X, A, B, G, a, b, e, i and R₁-R₇ are defined as follows:
X═O or H,OH (R or S) or H,OR (R or S) or H,H or H,NR (R or S); H,NRR′ (R or S); A, B, G, E, J, L═CH₂, C═O, CHOR (R or S); N, O, S wherein only chemically stable linkages are claimed (e.g., E═O,J═O is an acceptable peroxide if L contains stabilizing substituents at R₁₂ and/or R₁₃); A could also come from condensation with 1,3-dicarbonyls such that R_{1,2}═COMe or COOEt or combinations thereof;
b=0,1; e=0,1; i=0,1; a=1-6
R, R′, R₁-R₇═H or optionally substituted alkyl, axyl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO₂R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —CH₂)ₙNRR′; phenol, alkoxy (R′O), aryloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, furanyl, thiopheneyl, and benzo homologues (e.g. indolyl, quinolyl, isoquinolyl, etc.); and substituted variants therein, wherein any A, B, G that is potentially chiral can be a diastereomer or enantiomer, or diastereomeric mixtures and racemates.

8. A compound of the Formula 12: 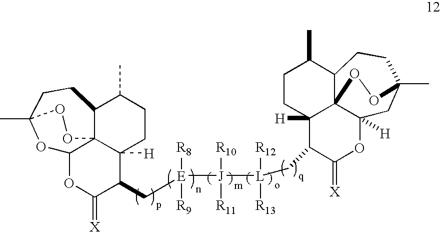
where X, E, J, L, p, q, n, m, o, and R₈-R₁₃ are defined as follows:
X═O or H,OH (R or S) or H,OR (R or S) or H,H or H,NR (R or S); H,NRR′ (R or S); E, J, L═CH₂, C═O, CHOR (R or S); N, O, S wherein only chemically stable linkages are claimed (e.g., E═O, J═O is an acceptable peroxide if L contains stabilizing substituents at R₁₂ and/or R₁₃). E or L could also come from condensation with 1,3-dicarbonyls such that R_{8,9} and or R_{12,13}═COMe or COOEt or combinations thereof;
n=0, 1; m=0, 1; o=0, 1; p,q=1-6
R, R₈-R₁₃═H or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include halogen; thiol, thioalkyl, sulfonylalkyl (SOR′), sulfone (SO₂R′), sulfonamido (NHSQ₂R′), acylsulfonamido (R″CONSO₂R′), N-alkylsulfonamido (R″NSO₂R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —CH₂)ₙNRR′; phenol, alkoxy (R′O), aryloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, furanyl, thiopheneyl, and benzo homologues (e.g. indolyl, quinolyl, isoquinolyl, etc.); and substituted variants therein, wherein any A, B, G that is potentially chiral can be a diastereomer or enantiomer, or diastereomeric mixtures and racemates;

9. A compound of the Formula 13 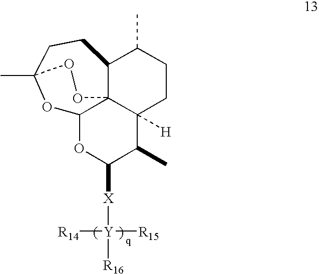
where X, Y and R₁₄-R₁₆ are defined as follows:
X═S, SO, SO₂, O, NH, NR, NOH, NOR, C═C, C═O, CH₂, Y═C, N, O, S, C═O, or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include, but are not limited to, halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO2R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂)NRR′; phenol, alkoxy (R′O), aryloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, furanyl, thiopheneyl, and benzo homologues (e.g. indolyl, quinolyl, isoquinolyl, etc.); and substituted variants therein;
q=0, 1, 2 etc,
R, R₁₄-R₁₆═H or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include, but are not limited to, halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO₂R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂)ₙNRR′; phenol, alkoxy (R′O), aryloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, furanyl, thiopheneyl, and benzo homologues (e.g. indolyl, quinolyl, isoquinolyl, etc.); and substituted variants therein and so on wherein any X and/or Y that is potentially chiral can be a diastereomer or enantiomer, or diastereomeric mixtures and racemates;

10. A compound of the Formula 14. 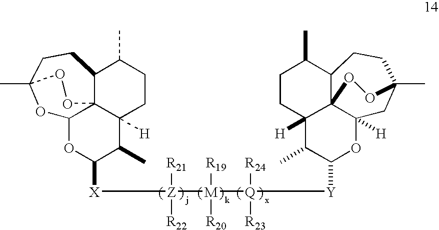
where X, Y, Z, M, Q, and R₂₁-R₂₄ are defined as follows:
X═S, SO, SO₂, O, C═O, CH₂,
Y═S, SO, SO₂, O, C═O, CH₂,
Z, M, Q═C, N, O, S, C═O, or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include, but are not limited to, halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO2R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂)ₙNRR′; phenol, alkoxy (R′O), aryloxy (ArO), haloalkyl; alkyl, alkenyl, arylalkyl, arylalkenyl, aryl; substituted aryl; heteroaryl; substituted heteroaryl. R is also heterocyclic and heteroaromatic, such as pyridyl, pyrrolyl, furanyl, thiopheneyl, and benzo homologues (e.g. indolyl, quinolyl, isoquinolyl, etc.); and substituted variants therein;
q=0, 1, 2 etc.
R, R₁₄-R₁₆═H or optionally substituted alkyl, aryl, heteroaryl, alkylaryl, alkylheterocyclic, heteroalkyl, alkylheteroaryl, cycloalkyl, alkylcycloalkyl, bicycloalkyl groups such as ortho, meta or para substituted benzenoids; where the substitutents include, but are not limited to, halogen; thiol, thioalkyl, sulfonylalkyl (SOR′), sulfone (SO₂R′), sulfonamido (NHSO₂R′), acylsulfonamido (R″CONSO₂R′), N-alkylsulfonamido (R″NSO₂R′), nitro, amino, N-alkyl or N-aryl or N-heteroaryl amino; N,N-dialkylamino (NR′R″), N-alkyl-N′-aryl (and N′-heteroaryl)amino, N,N′-diarylamino, N-aryl-N′-heteroarylamino and the corresponding homologated amines such as ortho, meta or para —(CH₂)ₙNRR′; phenol, alkoxy (R′O), aryloxy (ArO), haloalkyl; alkyl, alkenyl, arylalkyl, arylalkenyl, aryl; substituted aryl; heteroaryl; substituted heteroaryl; R is also heterocyclic and heteroaromatic, such as pyridyl, pyrrolyl, furanyl, thiopheneyl, and benzo homologues (e.g. indolyl, quinolyl, isoquinolyl, etc.); and substituted variants therein and so on wherein any X, Y, Z, M and/or Q that is potentially chiral can be a diastereomer or enantiomer, or diastereomeric mixtures and racemates.

11. A pharmaceutical composition comprising, said composition comprising at least one of any one of claims 7, 8, 9 or 10 and a pharmaceutically acceptable carrier or excipient.

12. A method of treating an infectious or cancerous disease sensitive to oxidative stress, said method comprising administering to a subject a therapeutically effective amount of at least one compound of the formulae of any one of claims 7, 8, 9, or 10.
